(19) United States
(12) Patent Application Publication
(10) Pub. No.: US 2024/0216553 A1
(43) Pub. Date: Jul. 4, 2024

(54) DEVELOPMENT OF CD163 TARGETING AGENT FOR IMAGING AND THERAPY

(71) Applicant: Washington University, St. Louis, MO (US)

(72) Inventors: Yongjian Liu, St. Louis, MO (US); Xiuli Zhang, St. Louis, MO (US); Gyu Seong Heo, St. Louis, MO (US)

(21) Appl. No.: 18/400,969

(22) Filed: Dec. 29, 2023

Related U.S. Application Data
(60) Provisional application No. 63/435,978, filed on Dec. 29, 2022.

(51) Int. Cl. A61K 51/08; A61K 51/04

(52) U.S. Cl. CPC A61K 51/088; A61K 51/0482; A61K 2121/00; A61K 2123/00

(57) ABSTRACT

Among the various aspects of the present disclosure is the provision of compositions of CD163 targeting and theranostic agents and methods for use in detecting, monitoring, and evaluating CD163 associated diseases, disorders, and conditions.

### CROSS-REFERENCE TO RELATED APPLICATIONS

This application claims the benefit of and incorporates by reference the content of U.S. Provisional App. No. 63/435,978, filed on Dec. 29, 2022.

### GOVERNMENT SUPPORT

This invention was made with government support under HL145212 and EB025815 awarded by the National Institutes of Health. The government has certain rights in the invention.

### SEQUENCE LISTING

The instant application contains a Sequence Listing which has been submitted electronically in XML format and is hereby incorporated by reference in its entirety. The computer readable file, created on Dec. 29, 2023, is named 047563_783244.xml and is about 4,356 bytes in size.

### FIELD OF THE TECHNOLOGY

The present invention relates to compositions and methods for non-invasive theranostic agent to track resident macrophages in vivo. More particularly, the invention relates to compositions and methods which aid in understanding the role of resident macrophages in the pathogenesis of various inflammatory diseases to promote both diagnosis and treatment.

### BACKGROUND

Cardiovascular disease (CVD) is the leading cause of death worldwide with atherosclerosis being the primary cause (1,2). It is known that macrophages play important and diverse roles in the initiation, progression, and complication of atherosclerosis (3), thus making them not only as diagnostic biomarkers to assess the progression and vulnerability of plaques (4-7), but also as targets in drug development for pharmacological intervention to modulate their activities in atherosclerosis and other inflammatory diseases (8-12).

Transformation of different phenotypes of macrophages regulates the initiation, development, and cessation of inflammatory diseases (13). Thus, it is critical to track the well-defined macrophage populations with constant expression of surface markers across multiple organs to interrogate their temporal-spatial distribution along the progression and regression of inflammatory diseases (14-16). Many molecular imaging agents have been developed to detect specific biomarkers (i.e., C—C motif chemokine receptor 2 (CCR2), CXC motif chemokine receptor 4 (CXCR4)) upregulated on macrophages to assess the vulnerability of atherosclerotic lesions (7,17-23). Although some of these agents have shown promising results in patients with atherosclerosis, their potential to predict the rupture of plaques has not been realized (23-25). The non-invasive and accurate identification of patients with high-risk plaques who may benefit from immunomodulatory therapies is still an unmet clinical need.

CD163 is a 130 kDa transmembrane scavenger receptor almost exclusively expressed by resident macrophages. In contrast to the infiltrating monocyte-derived macrophages that often accumulate in response to local inflammatory cues in the specific tissue, resident macrophages are seeded during embryonic development and reside in many tissues including liver and brain (14). The CD163+ macrophages play important role in tissue homeostasis and serve as first line defender for pathogen invasion due to their anti-inflammatory functions (11). Moreover, CD163 is known as the exclusive receptor to clear the hemoglobin-haptoglobin complex upon intravascular hemolysis. This leads to a distinct phenotype of macrophage termed M(Hb) found in areas of neoangiogenesis and hemorrhage, characterized by high expression of CD163 and reduced proinflammatory cytokine production, which distinguish them from foamy macrophages (26-28). Given the role of intra-plaque hemorrhage in plaque destabilization (29), CD163+ macrophage expression is associated with plaque vulnerability in human carotid plaques and is significantly higher in symptomatic patients compared to the asymptomatic population, highlighting its potential as a biomarker for vulnerable plaques (26,30).

The detection of CD163 could be of clinical significance not only to determine the vulnerability of atherosclerotic plaques, but also to provide critical information as a target to manage the treatment. However, there is no molecular agent available for CD163 targeting, which is an unmet need for both preclinical and clinical research.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

Provided herein is a CD163 targeting and theranostic composition. The CD163 targeting and theranostic composition may include a targeting peptide comprising an amino acid sequence, a chelator and a theranostic agent. The amino acid sequence may be any one of SEQ ID NO. 1, SEQ ID NO. 2, SEQ ID NO. 3, or SEQ ID NO. 4.

In some embodiments, the chelator may be NODAGA. In some embodiments, the theranostic agent may be ⁶⁴Cu. In some embodiments, the targeting peptide and chelator may be covalently linked and the theranostic agent may be a radionuclide.

In some embodiments, the amino acid sequence may be SEQ ID NO. 1, the chelator is NODAGA, and the theranostic agent is ⁶⁴Cu.

Provided herein is a method of detecting CD163 in a subject in need thereof. The method may include administering to the subject a composition comprising the disclosed CD163 targeting and theranostic composition and detecting the presence, amount, and/or location of the theranostic agent. The detecting of the theranostic agent may be indicative of CD163. The amino acid sequence may comprise SEQ ID NO. 1, SEQ ID NO. 2, SEQ ID NO. 3, or SEQ ID NO. 4.

In some embodiments of the method, the chelator may be NODAGA. In some embodiments, the theranostic agent may be ⁶⁴Cu. In some embodiments of the method, the targeting peptide and chelator may be covalently linked and the theranostic agent may be a radionuclide.

In some embodiments of the method, the amino acid sequence may be SEQ ID NO. 1, the chelator is NODAGA, and the theranostic agent is ⁶⁴Cu.

Provided herein is a method of detecting inflammation in a subject. The method may include administering to the subject a composition comprising the disclosed CD163 targeting and theranostic composition and detecting the presence, amount, and/or location of the theranostic agent. The detecting of the theranostic agent may be indicative of inflammation. The amino acid sequence may comprise SEQ ID NO. 1, SEQ ID NO. 2, SEQ ID NO. 3, or SEQ ID NO. 4.

In some embodiments of the method, the chelator may be NODAGA. In some embodiments, the theranostic agent may be ⁶⁴Cu. In some embodiments of the method, the targeting peptide and chelator may be covalently linked and the theranostic agent may be a radionuclide.

In some embodiments of the method, the amino acid sequence may be SEQ ID NO. 1, the chelator is NODAGA, and the theranostic agent is ⁶⁴Cu.

Provided herein is a method of detecting and/or monitoring atherosclerotic plaques in a subject. The method may include administering to the subject a composition comprising the disclosed CD163 targeting and theranostic composition and detecting the presence, amount, and/or location of the theranostic agent. The detecting of the theranostic agent may be indicative of atherosclerotic plaques. The amino acid sequence may comprise SEQ ID NO. 1, SEQ ID NO. 2, SEQ ID NO. 3, or SEQ ID NO. 4.

In some embodiments of the method, the chelator may be NODAGA. In some embodiments, the theranostic agent may be ⁶⁴Cu. In some embodiments of the method, the targeting peptide and chelator may be covalently linked and the theranostic agent may be a radionuclide.

Provided herein is a method of treating a cancer in a subject in need thereof. The method may include administering to the subject a composition comprising the disclosed CD163 targeting and theranostic composition and detecting the presence, amount, and/or location of the theranostic agent. The amino acid sequence may comprise SEQ ID NO. 1, SEQ ID NO. 2, SEQ ID NO. 3, or SEQ ID NO. 4. In some embodiments, the theranostic agent may be ²²⁵At. In some embodiments, the cancer may be a CD163 positive cancer. In some embodiments, the cancer may be a solid tumor. In some embodiments, the cancer may be ovarian cancer, melanoma, hepatocellular carcinoma, colorectal cancer, head and neck cancer, pancreatic cancer, breast cancer, stomach cancer, lung cancer, prostate cancer, colon cancer, brain cancer, or myloma.

### BRIEF DESCRIPTION OF THE FIGURES

The patent or application file contains at least one drawing executed in color. Copies of this patent or patent application with color drawing(s) will be provided by the Office by request and payment of the necessary fee.

FIG. 1 is a schematic illustration showing the dynamic variation of macrophage subsets during promotion of inflammation and inflammation resolution.

FIG. 2A shows PET images of [⁶⁴Cu]-DOTA-vMIP-II according to some embodiments of the present disclosure.

FIG. 2B shows PET images of [⁶⁴Cu]-DOTA-ECL1i PET images according to some embodiments of the present disclosure.

FIG. 2C show graphical representations of comparison of [⁶⁴Cu]-DOTA-vMIP-II to [⁶⁴Cu]-DOTA-ECL1i PET quantification in multiple mouse models including HNSCC, ApoE^{-/-} atherosclerosis and LPS induced lung inflammation.

FIG. 3A shows images of tumor imaging comparison among clinical CT, [¹⁸F]-FDG and [⁶⁴Cu]-DOTA-ECL1i PET (0-60 min dynamic scan) and DV images in HN-4 patient (green arrow: primary tumor; red arrow: lymph node).

FIG. 3B shows representative tumor TAC showing 1-comp (red) and 2-comp-reversible (best fit, blue) modeling with derived DV values.

FIG. 4A shows CT, PET and PET/CT at 0-60 dynamic scan and 1.5 h static scan of HN-4 at various views.

FIG. 4B shows PET/CT of HN-6 showing [⁶⁴Cu]-DOTA-ECL1i detecting primary tumors and lymph nodes (green arrow: primary tumor; red arrow: lymph node).

FIG. 4C shows tumor and Lymph node uptake (SUVₘₐₓ) and Target-to-background (SUVₘₐₓ) ratios for all 10 HNSCC patients at both time points.

FIG. 5A shows characterization of primary tumor and lymph node collected from HN-4. H&E shows a well-differentiated keratinizing SCC tumor with infiltration of lymphocytes. Immunofluorescent staining shows intense signals for CD68 (red) and CCR2 (green) across the tissue. Magnified region 1 shows that most tumor cells specifically expressing CCR2 while region 2 demonstrated the infiltration of CD68 positive TAMs with many positive for CCR2+(CD68+CCR2+: yellow, white arrow). Ex vivo autoradiography shows intensive binding of [⁶⁴Cu]-DOTA-ECL1i to the tissue. The competitive blocking and autoradiography quantification (n=8) demonstrated the tracer binding specificity. The correlation assay between autoradiography and CCR2 immunostaining indicated the tissue binding was CCR2 specific.

FIG. 5B shows characterization of primary tumor and lymph node collected from HN-4. H&E and CCR2 immunostaining revealed the intensive and specific expression of CCR2 in the tumor cells of lymph node. *** p<0.001.

FIG. 6 is a graphical representation of ELISA assay of screened peptides binding profiles (n=3/group). ** p<0.01, *** p<0.001, **** p<0.0001

FIG. 7 shows a comparison of peptide docking and predicted binding affinities (top 10 poses) to CCR2 between P8, P15, P21 and ECL1i.

FIG. 8 is a synthetic scheme and 18F labeling of AZ889.

FIG. 9 shows a synthetic scheme of NODAGA-ICT-01 synthesis according to some embodiments of the present disclosure.

FIG. 10A shows a graphical representation of in vitro cell binding assay of ⁶⁴Cu-NODAGA-ICT-01 in U87 cells according to some embodiments of the present disclosure.

FIG. 10B shows a bar graph of biodistribution of ⁶⁴Cu-NODAGA-ICT-01 in WT C57BL/6 mice (n=4/group) at 1, 2, and 4 hours post injection according to some embodiments of the present disclosure

FIG. 11 shows PET scan images of [⁶⁴Cu]-NODAGA-ICT-01 and [¹⁸F]-FDG PET in ApoE^{-/-} mice (n=4/group). * p<0.05, ** p<0.01, *** p<0.001.

FIG. 12 shows [⁶⁴Cu]-NODAGA-ICT-01 and [¹⁸F]-FDG PET/CT imaging in HNSCC MOC1 xenograft and PDX tumor models. H&E and immunofluorescent staining showed the infiltration of inflammatory cells that were positive for CD163. Purple circle: tumor.

FIGS. 13A-E show images of [⁶⁸Ga]-DOTA-ECL1i and [⁶⁸Ga]-NODAGA-ICT-01 imaging M1 CCR2+ and M2 CD163+ macrophages dynamics post MI in a mouse ischemia reperfusion injury model (n=4/group). Yellow arrow and orange arrowhead: infarct, white arrow head: remote zone. As shown in FIG. 13A, [¹⁸F]-FDG identified the infarct at day 3 post MI. Day 4 CCR2 PET revealed intensive tracer uptake at infarct zone, which gradually decreased through the remodeling process while the tracer accumulation at remote area remained stable during the study (FIG. 13B and FIG. 13 C). Interestingly, CD163 PET signals were observed at remote area and gradually increased from day 5 to day 13 and then remained stable until day 25 (FIG. 13D, FIG. 13E).

FIG. 14 is a bar graph showing peptide sequences identified from phase display showing binding to CD163(n=3) according to some embodiments of the present disclosure.

FIG. 15 is a bar graph showing peptide ELISA of biotinlated CD163 peptides (0.1 mg/mL) showing binding to CD163 (n=3) according to some embodiments of the present disclosure.

FIG. 16 is a bar graph showing the biodistribution of ⁶⁸Ga-ICT-01 at 1 hour post injection according to some embodiments of the present disclosure.

FIG. 17 shows PET/CT images and quantification of ⁶⁴Cu-ICT-01 in wild type mice, ApoE^{-/-} mice at 10 and 18 weeks post high fat diet according to some embodiments of the present disclosure. **** p<0.001.

FIG. 18 shows PET/CT images and quantification of ⁶⁴Cu-ICT-01 in wild type mice, HNSCC PDX 11c mice at 7 weeks post tumor implantation according to some embodiments of the present disclosure. T: tumor. * p<0.05.

FIG. 19A is a graphical representation of in vitro cell binding assay of ⁶⁴Cu-ICT-01 in human U87 cells and mouse 264.7 cells according to some embodiments of the present disclosure.

FIG. 19B is a graphical representation of in vivo biodistribution in wild type C57BL/6 mice (n=4/group) of ⁶⁴Cu-ICT-01 according to some embodiments of the present disclosure.

FIG. 20A shows PET/CT images of ⁶⁴Cu-ICT-01 in ApoE^{-/-} mice according to some embodiments of the present disclosure. * p<0.05, ** p<0.01, *** p<0.001, **** p<0.0001.

FIG. 20B is a graphical representation of quantification of tracer uptake at aortic arches. in WT and ApoE^{-/-} mice on HFD at various time points (n=4-7/group) according to some embodiments of the present disclosure. * p<0.05, ** p<0.01, *** p<0.001, **** p<0.0001.

FIG. 20C shows H&E and immunofluorescent staining images of ⁶⁴Cu-ICT-01 in ApoE^{-/-} mice according to some embodiments of the present disclosure.

FIG. 20D show ⁶⁴Cu-ICT-01 autoradiography images of aorta, spleen, and thymus.

FIG. 20E shows flow cytometry data of CD163+ macrophages in ApoE^{-/-} mice according to some embodiments of the present disclosure.

FIG. 20F shows scRNAseq of macrophages collected from ApoE^{-/-} mouse aortas after 20 weeks on HFD. Resident macrophage gene set score using FoIr2, Lyve1, and F13a1 (top) and gene expression density for Cd163 in UMAP embedding according to some embodiments of the present disclosure.

FIG. 21A shows PET images of ⁶⁴Cu-ICT-01 in PCSK9 mice according to some embodiments of the present disclosure.

FIG. 21B is a graphical representation of quantification of tracer uptake at aortic arches at various time points on HFD (n=4-8/group) according to some embodiments of the present disclosure.

FIG. 22A shows ex vivo characterization of human CEA specimen according to some embodiments of the present disclosure. H&E shows an advanced plaque with large fibrous core, IPH (asterisk, green box and magnified image), calcification, and infiltration of inflammatory cells. CD163 immunofluorescent staining show extensive expression of CD163 across the tissue with most signals associated with IPH (green box and magnified image), and inflammatory cells.

FIG. 22B illustrates that ⁶⁴Cu-ICT-01 autoradiography shows high binding to the CEA tissues according to some embodiments of the present disclosure. Blocking revealed significantly decreased signals, suggesting binding specificity. Both ROIs and whole tissue analyses on the coregistered autoradiography and immunofluorescent staining showed good correlation.

FIG. 23 is a schematic illustration of a scheme of ICY-01 conjugation with NODAGA chelator according to some embodiments of the present disclosure.

FIG. 24 is a graphical representation of the biodistribution and blocking of ⁶⁴Cu-ICT-01 in wild type C57BL/6 mice (n=4 group) * p<0.05, ** p<0.01, **** p<0.0001.

FIG. 25A is an image of the maximum intensity projection images of ⁶⁴Cu-ICT-01 in ApoE^{-/-} mice on HFD for 40 weeks of according to some embodiments of the present disclosure.

FIG. 25B is an image of the maximum intensity projection images of ⁶⁴Cu-ICT-01 in PCSK9 mice at 29 weeks after HFD according to some embodiments of the present disclosure.

FIG. 26A is an image showing a CT and ⁶⁴Cu-ICT-01 PET/CT in an ApoE^{-/-} mouse at 40 weeks post HFD shows a strong PET signal associated with microcalcification within aortic arch according to some embodiments of the present disclosure.

FIG. 26B is a graphical representation of Hounsfield units measurement of WT mice (n=7/group), ApoE^{-/-} mouse at 10 wk (n=7/group) and 40 weeks (n=4/group) post HFD showed increased values overtime according to some embodiments of the present disclosure. *** p<0.001.

FIG. 27A is an image showing ⁶⁴Cu-ICT-01 PET/CT in a Ldlr^{-/-} mouse at 30 weeks post HFD shows a strong PET signal within aoric arch according to some embodiments of the present disclosure.

FIG. 27B is a graphical representation showing significantly increased uptake at aortic arch of Ldlr^{-/-} mouse (n=6/group) than that in WT mice (n=7/group) according to some embodiments of the present disclosure. ** p<0.01

FIG. 28 shows images illustrating control immunofluorescent staining of mouse aorta collected from ApoE^{-/-} after 20 weeks' HFD according to some embodiments of the present disclosure.

FIG. 29A is a graphical representation of a correlation analysis between ⁶⁴Cu-ICT-01 uptake at aortic arch and numbers of CD163+ macrophages/500 μm² according to some embodiments of the present disclosure.

FIG. 29B is a graphical representation of a correlation analysis between plaque size in ApoE^{-/-} KO mice and numbers of CD163+ macrophages/500 μm² according to some embodiments of the present disclosure.

FIG. 29C is a graphical representation of a correlation analysis between between ⁶⁴Cu-ICT-01 uptake at aortic arch and plaque size in ApoE^{-/-} KO mice according to some embodiments of the present disclosure.

FIG. 30 shows flow cytometry gating strategy for CD163 analysis in ApoE^{-/-} mice according to some embodiments of the present disclosure.

FIG. 31 shows ScRNAseq of all immune cells in digested ApoE^{-/-} mouse aortas according to some embodiments of the present disclosure. The monocytes derived macrophages shows a distinct distribution from tissue resident macrophages.

FIG. 32A shows H&E and CD163 immunofluorescent staining of aortic arteries from LDLR KO mice at 37-week post HFD according to some embodiments of the present disclosure.

FIG. 32B shows H&E and CD163 immunofluorescent staining of aortic arteries from PCSK9 mice at 34-week post HFD according to some embodiments of the present disclosure.

FIG. 33 shows images of H&E and immunofluorescent staining of human CEA specimens according to some embodiments of the present disclosure.

### DETAILED DESCRIPTION

The present invention provides compositions useful for targeting and imaging CD163. The compositions generally include a targeting peptide, a chelator or a nanoparticle or a linker, and an imaging agent. The compositions are useful for non-invasive imaging to track resident macrophages in vivo. More particularly, the invention relates to compositions and methods which aid in understanding the role of resident macrophages in the pathogenesis of various inflammatory diseases to promote both diagnosis and treatment.

Various aspects of the invention are described in further detail in the following sections.

### CD163 Targeting and Imaging Compositions

The compositions generally include a targeting peptide, a chelator or a nanoparticle or a linker, and an imaging agent.

### (a) Targeting Peptide

In some embodiments, targeting peptides may be used to target CD163 on a subtype of tissue resident macrophagesspecific proteins expressed on macrophages.

As described herein, the targeting and imaging agent comprises a CD163 binding peptide. The CD163 binding peptide can be any peptide with CD163 activity. The CD163 binding peptide as described herein can comprise an amino acid length of about 4 amino acids to about 100 amino acids or about 4 amino acids to about 50 amino acids. For example, the CD163 binding peptide can comprise an amino acid length of no more than 4 amino acids; 5 amino acids; 6 amino acids; 7 amino acids; 8 amino acids; 9 amino acids; 10 amino acids; 11 amino acids; 12 amino acids; 13 amino acids; 14 amino acids; 15 amino acids; 16 amino acids; 17 amino acids; 18 amino acids; 19 amino acids; 20 amino acids; 21 amino acids; 22 amino acids; 23 amino acids; 24 amino acids; 25 amino acids; 26 amino acids; 27 amino acids; 28 amino acids; 29 amino acids; 30 amino acids; 31 amino acids; 32 amino acids; 33 amino acids; 34 amino acids; 35 amino acids; 36 amino acids; 37 amino acids; 38 amino acids; 39 amino acids; 40 amino acids; 41 amino acids; 42 amino acids; 43 amino acids; 44 amino acids; 45 amino acids; 46 amino acids; 47 amino acids; 48 amino acids; 49 amino acids; 50 amino acids; 51 amino acids; 52 amino acids; 53 amino acids; 54 amino acids; 55 amino acids; 56 amino acids; 57 amino acids; 58 amino acids; 59 amino acids; 60 amino acids; 61 amino acids; 62 amino acids; 63 amino acids; 64 amino acids; 65 amino acids; 66 amino acids; 67 amino acids; 68 amino acids; 69 amino acids; 70 amino acids; 71 amino acids; 72 amino acids; 73 amino acids; 74 amino acids; 75 amino acids; 76 amino acids; 77 amino acids; 78 amino acids; 79 amino acids; 80 amino acids; 81 amino acids; 82 amino acids; 83 amino acids; 84 amino acids; 85 amino acids; 86 amino acids; 87 amino acids; 88 amino acids; 89 amino acids; 90 amino acids; 91 amino acids; 92 amino acids; 93 amino acids; 94 amino acids; 95 amino acids; 96 amino acids; 97 amino acids; 98 amino acids; 99 amino acids; or 100 amino acids. Recitation of each of these discrete values is understood to include ranges between each value. Recitation of each of a range is understood to include discrete values within the range.

Targeting peptides may be determined by any suitable technique. Examples of suitable techniques include but are not limited to two-hybrid systems, phage display, in silico approaches, or a combination thereof.

In some embodiments, the targeting peptide may be determined by phage display screening.

In some embodiments, the targeting peptide may be a 12-mer peptide. Table 1 below lists some CD163 binding peptides according to the present disclosure.

TABLE 1
CD163 Binding Peptides

| SEQ ID | | Decription of the |
|---|---|---|
| NO: | Name | peptide sequence |
| 1 | ICT-01 | WFWNHGWQWSSK |
| 2 | P08 | VDDHYGTIVRNG |
| 3 | P15 | HWKWLKPWAVNT |
| 4 | P21 | LIPAPGGTPRAG |

Each amino acid sequence described herein by virtue of its identity or similarity percentage with a given amino acid sequence respectively has in a further preferred aspect an identity or a similarity of at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% with the given nucleotide or amino acid sequence, respectively. The terms “homology”, “sequence identity” ^{m} and the like are used interchangeably herein. Sequence identity is described herein as a relationship between two or more amino acid (polypeptide or protein) sequences or two or more nucleic acid (polynucleotide) sequences, as determined by comparing the sequences. In a preferred aspect, sequence identity is calculated based on the full length (in amino acids or nucleotides) of two given SEQ ID NOS or based on a portion thereof. A portion of a full-length sequence may be referred to as a fragment, and preferably means at least 50%, 60%, 70%, 80%, 90%, or 100% of the length (in amino acids or nucleotides) of a reference sequence. “Identity” also refers to the degree of sequence relatedness between two amino acid sequences, or between two nucleic acid sequences, as the case may be, as determined by the match between strings of such sequences. The degree of sequence identity between two sequences can be determined, for example, by comparing the two sequences using computer programs commonly employed for this purpose, such as global or local alignment algorithms. Non-limiting examples include BLASTp, BLASTn, Clustal W, MAFFT, Clustal Omega, AlignMe, Praline, GAP, BESTFIT, or another suitable method or algorithm. A Needleman and Wunsch global alignment algorithm can be used to align two sequences over their entire length or part thereof (part thereof may mean at least 50%, 60%, 70%, 80%, 90% of the length of this sequence), maximizing the number of matches and minimizes the number of gaps. Default settings can be used and preferred program is Needle for pairwise alignment (in an aspect, EMBOSS Needle 6.6.0.0, gap open penalty 10, gap extent penalty: 0.5, end gap penalty: false, end gap open penalty: 10, end gap extent penalty: 0.5 is used) and MAFFT for multiple sequence alignment (in an aspect, MAFFT v7Default value is: BLOSUM62 [b162], Gap Open: 1.53, Gap extension: 0.123, Order aligned, Tree rebuilding number: 2, Guide tree output: ON [true], Max iterate: 2, Perform FFTS: none is used).

“Similarity” between two amino acid sequences is determined, for example, by comparing the amino acid sequence and its conserved amino acid substitutes of one polypeptide to the sequence of a second polypeptide. Similar algorithms used for determination of sequence identity may be used for determination of sequence similarity. Optionally, in determining the degree of amino acid similarity, the skilled person may also take into account so-called conservative amino acid substitutions. As used herein, “conservative” amino acid substitutions refer to the interchangeability of residues having similar side chains. Examples of classes of amino acid residues for conservative substitutions are given in the Tables below:

| | |
|---|---|
| Acidic Residues | Asp (D) and Glu (E) |
| Basic Residues | Lys (K), Arg (R), and His (H) |
| Hydrophilic Uncharged Residues | Ser (S), Thr (T), Asn (N), and |
| | Gln (Q) |
| Aliphatic Uncharged Residues | Gly (G), Ala (A), Val (V), Leu (L), |
| | and Ile (I) |
| Non-polar Uncharged Residues | Cys (C), Met (M), and Pro (P) |
| Aromatic Residues | Phe (F), Tyr (Y), and Trp (W) |

### Alternative Conservative Amino Acid Residue Substitution Classes:

| | | | |
|---|---|---|---|
| 1 | A | S | T |
| 2 | D | E | |
| 3 | N | Q | |
| 4 | R | K | |
| 5 | I | L | M |
| 6 | F | Y | W |

### Alternative Physical and Functional Classifications of Amino Acid Residues:

| | |
|---|---|
| Alcohol group-containing residues | S and T |
| Aliphatic residues | I, L, V, and M |
| Cycloalkenyl-associated residues | F, H, W, and Y |
| Hydrophobic residues | A, C, F, G, H, I, L, M, R, T, V, W, |
| | and Y |
| Negatively charged residues | D and E |
| Polar residues | C, D, E, H, K, N, Q, R, S, and T |
| Positively charged residues | H, K, and R |
| Small residues | A, C, D, G, N, P, S, T, and V |
| Very small residues | A, G, and S |
| Residues involved in turn | A, C, D, E, G, H, K, N, Q, R, S, P |
| formation | and T |
| Flexible residues | Q, T, K, S, G, P, D, E, and R |

For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to Ser; Arg to Lys; Asn to Gln or His; Asp to Glu; Cys to Ser or Ala; Gln to Asn; Glu to Asp; Gly to Pro; His to Asn or Gln; Ile to Leu or Val; Leu to Ile or Val; Lys to Arg; Gln or Glu; Met to Leu or lie; Phe to Met, Leu or Tyr; Ser to Thr; Thr to Ser; Trp to Tyr; Tyr to Trp or Phe; and, Val to Ile or Leu.

The “percent identity” of two amino acid sequences can be determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

As used herein “fragment” means a portion of a polypeptide that contains, preferably, at least 10%, 20%, 30%, 40%, 50%, 60%, 70%, 80%, 90%, 95%, or more of the entire length of the reference polypeptide. In certain aspects, the fragment as the same or similar biological activity as the reference polypeptide.

### (b) Imaging Agent

The imaging agent, as described herein, comprises a radiolabel (also known as a radionuclide). Radioactive forms of elements are called radionuclides. Some occur naturally in the environment, while others are man-made, either deliberately or as byproducts of nuclear reactions.

References herein to “radiolabeled” include a compound where one or more atoms are replaced or substituted by an atom having an atomic mass or mass number different from the atomic mass or mass number typically found in nature (i.e., naturally occurring). One non-limiting exception is ¹⁹F, which allows detection of a molecule which contains this element without enrichment to a higher degree than what is naturally occurring. Compounds carrying the substituent ¹⁹F may thus also be referred to as “labelled” or the like. The term radiolabeled may be interchangeably used with “isotopically-labelled”, “labelled”, “isotopic tracer group”, “isotopic marker”, “isotopic label”, “detectable isotope”, or “radioligand”.

Every radionuclide emits radiation at its own specific rate, which is measured in terms of half-life. Radioactive half-life is the time required for half of the radioactive atoms present to decay. Radioactive decay is when a radioisotope transforms into another radioisotope; this process emits radiation in some form. Some radionuclides have half-lives of mere seconds, but others have half-lives of millions of years.

The half-life of the radionuclides may be 1 hour to about 20 hours. For example, the half-life may be about 1 hour, about 1.5 hours, about 2 hours, about 2.5 hours, about 3 hour, about 3.5 hours, about 4 hours, about 4.5 hours, about 5 hour, about 5.5 hours, about 6 hours, about 6.5 hours, about 7 hour, about 7.5 hours, about 8 hours, about 8.5 hours, about 9 hours, about 9.5 hours, 10 hour, about 10.5 hours, about 11 hours, about 11.5 hours, about 12 hours, about 12.5 hours, about 13 hour, about 13.5 hours, about 14 hours, about 14.5 hours, about 15 hour, about 15.5 hours, about 16 hours, about 16.5 hours, about 17 hour, about 17.5 hours, about 18 hours, about 18.5 hours, about 19 hours, about 19.5 hours, or about 20 hours. The half-life may be between about 1 hour to about 20 hours, about 2 hours to about 19 hours, about 3 hours to about 18 hours, about 4 hours to about 17 hours, about 5 hours to about 16 hours, about 6 hours to about 15 hours, about 7 hours to about 14 hours, about 8 hours to about 13 hours, about 9 hours to about 12 hours, or about 10 hours to about 11 hours.

Radiolabeling processes are well known (Fani et al. Theranostics 2012; 2(5):481-501). Except as otherwise noted herein, therefore, the process of the present disclosure can be carried out in accordance with such processes.

One embodiment of the present disclosure provides for a radiolabeled peptide.

According to another embodiment, the radiolabeled compound can be an imaging agent.

In one embodiment, the compound comprises one or more radiolabeled groups.

Examples of suitable, non-limiting radiolabel groups can include: ²H (D or deuterium), ³H (T or tritium), ¹¹C, ¹³C, ¹⁴C, ⁶⁴Cu, ⁶⁷Cu, ¹⁷⁷Lu, ¹³N, ¹⁵N, ¹⁵O, ¹⁷O, ¹⁸O, ¹⁸F, ⁸⁹Sr, ³⁵S, ⁴⁴Sc, ⁵²Mn, ¹⁵³Sm, ³⁶Cl, ⁶²Br, ⁷⁵Br, ⁷⁶Br, ⁷⁷Br, ⁸⁶Y, ¹²³I, ¹²⁴I, ¹²⁵I, ¹³¹I, ¹¹¹In, ⁶⁷Ga, ⁶⁸Ga, ¹⁷⁷Lu, ¹⁸⁶Re, ¹⁶³Re, ²²⁵At, ²²³Ra, ²²⁷Th, ²¹¹At, ²¹²Bi, ²¹²Pb, ²¹³Bi, ²²⁴Ra, ²⁰¹Tl, ^{99m}Tc, ⁹⁰Y, or ⁸⁹Zr. It is to be understood that an isotopically labeled compound needs only to be enriched with a detectable isotope to, or above, the degree which allows detection with a technique suitable for the particular application, e.g., in a detectable compound labeled with ¹¹C, the carbon-atom of the labeled group of the labeled compound may be constituted by ¹²C or other carbon-isotopes in a fraction of the molecules. The radionuclide that is incorporated in the radiolabeled compounds will depend on the specific application of that radiolabeled compound. For example, “heavy” isotope-labeled compounds (e.g., compounds containing deuterons/heavy hydrogen, heavy nitrogen, heavy oxygen, heavy carbon) can be useful for mass spectrometric and NMR based studies. As another example, for in vitro labelling or in competition assays, compounds that incorporate ³H, ¹⁴C, or ¹²⁵I can be useful. For in vivo imaging applications ¹¹C, ¹³C, ¹⁸F, ¹⁹F, ¹²⁰I, ¹²³I, ¹³¹I, ⁷⁵Br, or ⁷⁶Br can generally be useful. In one embodiment, the radiolabel is 64Cu.

As another example, the imaging agent comprising a radiolabel can comprise Oxygen-15 water, Nitrogen-13 ammonia, [⁸²Rb] Rubidium-82 chloride, [¹¹C], [¹¹C] 25B-NBOMe, [¹⁸F] Altanserin, [¹¹C] Carfentanil, [¹¹C] DASB, [¹¹C] DTBZ, [¹⁸F]Fluoropropyl-DTBZ, [¹¹C] ME@HAPTHI, [¹⁸F] Fallypride, [¹⁸F] Florbetaben, [¹⁸F] Flubatine, [¹⁸F]Fluspidine, [¹⁸F] Florbetapir, [¹⁸F] or [¹¹C] Flumazenil, [¹⁸F] Flutemetamol, [¹⁸F] Fluorodopa, [¹⁸F] Desmethoxyfallypride, [¹⁸F] Mefway, [¹⁸F] MPPF, [¹⁸F] Nifene, Pittsburgh compound B, [¹¹C] Raclopride, [¹⁸F] Setoperone, [¹⁸F] or [¹¹C] N-Methylspiperone, [¹¹C] Verapamil, [¹¹C] Martinostat, Fludeoxyglucose (¹⁸F)(FDG)-glucose analogue, [¹¹C] Acetate, [¹¹C] Methionine, [¹¹C] Choline, [¹⁸F] Fluciclovine, [¹⁸F] Fluorocholine, [¹⁸F] FET, [¹⁸F] FMISO, [¹⁸F] 3′-fluoro-3′-deoxythymidine, [⁶⁸Ga] DOTA-pseudopeptides, [⁶⁸Ga] PSMA, or [¹⁸F] Fluorodeoxysorbitol (FDS).

### (c) Chelator

Chelation is a type of bonding of ions and molecules to metal ions. It involves the formation or presence of two or more separate coordinate bonds between a polydentate (multiple bonded) ligand and a single central metal atom

Suitable chelators for the invention include chelators that attach the targeting peptide to the imaging agent. Chelators generally work by binding to metal ions. As described herein, radionuclides can be chelated by any method known in the art.

Processes of chelating a radioligand are well known (e.g. Anderson et al., Cancer Biother Radiopharm. 2009 August; 24(4): 379-393; Stockholf et al., Pharmaceuticals (Basel). 2014 April; 7(4): 392-418). Except as otherwise noted herein, therefore, the process of the present disclosure can be carried out in accordance with such processes. For example, chelators for a radiolabel (e.g., ⁶⁴Cu) can be any of those known in the art (e.g., a macrocyclic chelator). As another example, the chelator can comprise NHS-MAG3, MAG3, DTPA, 3p-C-NE3TA, 3p-C-NOTA, 3p-C-DE4TA, ATSM, tetraazamacrocyclic ligands (e.g., DOTA (1,4,7,10-tetraazacyclododecane-1,4,7,10-tetraacetic acid), DOTA-NHS, pSCN-Bn-DOTA, pNH2-Bn-DOTA, TETA (1,4,8,11-tetraazacyclotetradecane-1,4,8,11-tetraacetic acid, TETA-octreotide (OC)), hexaazamacrobicyclic cage-type ligands (e.g., Sarcophogine chelators), cross-bridged tetraamine ligands (e.g., CB-TE2A (4,11-bis(carboxymethyl)-1,4,8,11-tetraazabicyclo[6.6.2]hexadecane)), 6-Hydrazinopridine-3-carboxylic acid (Hynic), or NHS-Hynic. As another example, a radiolabelled (e.g., ⁶⁴Cu) chelator can be 2,2′,2″-(10-(2-((2-(2,5-dioxo-2,5-dihydro-1H-pyrrol-1-yl)ethyl)amino)-2-oxoethyl)-1,4,7,10-tetraazacyclododecane-1,4,7-triyl)triacetic acid (Maleimido-mono-amide-DOTA).

The targeting peptide and chelator may be covalently linked. The chelator may be attached to the targeting peptide N- or C-terminus. In some embodiments, the chelator may be attached to the peptide through the N-terminus. In some embodiments, the chelator may be attached to the peptide through the C-terminus.

In some embodiments, the chelator may include 2-[1,4,7-Tiazacyclononan-1-yl-4,7-bis(tBu-ester)]-1,5-pentanedioic acid (NODAGA), DOTA, or any other suitable chelator.

### (d) Nanoparticle

As described herein, a radiolabel can be doped in or on a nanoparticle, or a radiolabel can be conjugated to a nanoparticle.

The imaging agent, as described herein can comprise any nanoparticle known in the art suitable for use as an imaging agent. Nanoparticles for use in molecular probes and imaging agents are well known; see e.g., Chen et al., Molecular Imaging Probes for Cancer Research, 2012.

Labeling of nanoparticles are well known; see e.g., Yongjian Liu, Michael J Welch, Nanoparticles labeled with positron emission nuclides: advantages, methods, and applications, Bioconjugate Chemistry, 2012, 23, 671-682; Stockholf et al., Pharmaceuticals (Basel). 2014 April; 7(4): 392-418. Except as otherwise noted herein, therefore, the process of the present disclosure can be carried out in accordance with such processes.

For example, a nanoparticle can be a nanocluster or any other type of nanostructures including organic, inorganic, or lipid nanostructures.

As another example, the nanoparticle can comprise Au or Cu. As another example, the nanoparticle can comprise iron oxide, gold, gold nanoclusters (AuNC), gold nanorods (AuNR), copper (Cu), quantum dots, carbon nanotubes, carbon nanohom, gadolinium (Gd), dendrimers, dendrons, polyelectrolyte complex (PEC) nanoparticles, calcium phosphate nanoparticles, perfluorocarbon nanoparticles (PFCNPs), or lipid-based nanoparticles, such as liposomes and micelles.

### (e) Linker

Described herein are linkers used to attach peptides to a portion of an imaging agent (e.g., a core, a nanoparticle, a radiolabel, a chelator, another peptide). A linker can be any composition used for conjugation, for example to a nanoparticle or chelator.

A linker group can be any linker group suitable for use in an imaging agent. Linker groups for imaging agents (e.g., molecular probes) are well known (see e.g., Werengowska-Ciedwierz et al., Advances in Condensed Matter Physics, Vol. 2015 (2015); Chen et al., Curr Top Med Chem. 2010; 10(12): 1227-1236). Except as otherwise noted herein, therefore, the processes of the present disclosure can be carried out in accordance with such processes.

For example, the linker can conjugate a nanoparticle to a CCR2 biding peptide. For example, the CCR2 binding peptide can be covalently attached to the linker. For example, the linker can comprise a poly(ethylene glycol) (PEG) derivative. As another example, the linker can comprise PEG, TA-PEG-Maleimide, TA-PEG-OMe, or TA-PEG. As another example, a linker can comprise an isothiocyanate group, a carboxylic acid or carboxylate groups, a dendrimer, a dendron, Fmoc-protected-2,3-diaminopropanoic acid, ascorbic acid, a silane linker, minopropyltrimethoxysilane (APTMS), or dopamine. Other covalent coupling methods can use employ the use of 2 thiol groups, 2 primary amines, a carboxylic acid and primary amine, maleimide and thiol, hydrazide and aldehyde, or a primary amine and aldehyde. For example, the linker can be an amide, a thioether, a disulfide, an acetyl-hydrazone group, a polycyclic group, a click chemistry (CC) group (e.g., cycloadditions, for example, Huisgen catalytic cycloaddition; nucleophilic substitution chemistry, for example, ring opening of heterocyclic electrophiles; carbonyl chemistry of the “nonaldol” type, for example, formation of ureas, thioureas, and hydrazones; additions to carbon-carbon multiple bonds, for example, epoxidation and dihydroxylation); or a physical or chemical bond,

### Method of Making Cd163 Targeting and Imaging Composition

Aspects of the present disclosure also relate to a method of making a CD163 targeting and imaging composition. The method generally includes conjugating a chelator or linker or nanoparticle with a CD163 targeting peptide to form a targeted conjugate followed by radiolabeling the targeted conjugate with a radionuclide to form the CD163 targeting and imaging composition.

In some embodiments, the chelator may be a macrocyclic chelator. Examples of a macrocyclic chelator includes but is not limited to 2-[1,4,7-Triazacyclononan-1-yl-4,7-bis(tBu-ester)]-1,5-pentanedioic acid (NODAGA). The chelator may be conjugated to the targeting peptide via amide bond. The amide bond may be between a tryptophan residue on N-terminus.

In some embodiments, the chelator may be conjugated to the N-terminus of the peptide via an amide bond. The conjugation may be performed in the presence of a tertiary amine and a suitable solvent. The conjugation may be further performed in the presence of a reagent used in peptide coupling chemistry to generate an active ester from a carboxylic acid. In some embodiments, the tertiary amine may be N,N-diisopropylethylamine. In some embodiments, the solvent may be DMF. In some embodiments, the reagent may be HATU. The first step of the synthesis may include combining the chelator, tertiary amine, solvent, and peptide coupling reagent at room temperature to form a chelator solution. The peptide may be added to the chelator solution and incubated overnight. The chelator conjugated to the peptide may be removed from the reaction mixture by appropriate separation techniques.

The radiolabeling of the chelator conjugate may be carried out using an appropriate radionuclide in the presence of a suitable buffer. In some embodiments, the radionuclide may be copper-64, gallium-68, or a combination thereof.

### Methods of Use

Methods described herein are generally performed on a subject in need thereof. A subject in need of the evaluation or therapeutic methods described herein can be a subject having, diagnosed with, suspected of having, or at risk for developing a CD163 associated disease, disorder, or condition. The subject in need may also be at risk for developing resident macrophage mediated inflammatory diseases including but not limited to CVD, atherosclerosis, including atherosclerosis vulnerable plaques. A determination of the need for treatment will typically be assessed by a history and physical exam consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and chickens, and humans. For example, the subject can be a human subject.

Aspects of the present disclosure relate to a method of detecting CD163 in a subject. The method includes administering to the subject a composition comprising the CD163 targeting and imaging agent described above and detecting the presence, amount, and/or location of the imaging agent. The detection of the imaging agent may be indicative of CD163.

Aspects of the present disclosure also relate to a method of detecting activated macrophases in a subject. The method includes administering to the subject a composition comprising the CD163 targeting and imaging agent described above and detecting the presence, amount, and/or location of the imaging agent. The detection of the imaging agent may be indicative of activated macrophases.

Aspects of the present disclosure further relate to a method of detecting inflammation in a subject. The method includes administering to the subject a composition comprising the CD163 targeting and imaging agent described above and detecting the presence, amount, and/or location of the imaging agent. The detection of the imaging agent may be indicative of inflammation.

Aspects of the present disclosure further relate to a method of monitoring atherosclerotic plaques in a subject. The method includes administering to the subject a composition comprising the CD163 targeting and imaging agent described above and detecting the presence, amount, and/or location of the imaging agent. The detection of the imaging agent may be indicative of atherosclerotic plaques.

The methods of the invention may also include treatment of any diseases or conditions resulting from resident macrophage mediated inflammatory diseases. The treatment may include those known to a skilled artisan such as immunomodulatory therapies or other therapies detailed herein.

### Formulations

A pharmaceutical composition of the invention may be formulated to be compatible with its intended route of administration. Examples of routes of administration include parenteral, e.g., intravenous, intradermal, subcutaneous, and rectal administration. Solutions or suspensions used for parenteral or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use may include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL (BASF; Parsippany, N.J.), or phosphate buffered saline (PBS). In all cases, a composition may be sterile and may be fluid to the extent that easy syringeability exists. A composition may be stable under the conditions of manufacture and storage and may be preserved against the contaminating action of microorganisms such as bacteria and fungi. The carrier may be a solvent or dispersion medium containing, for example, water, ethanol, polyol (for example, glycerol, propylene glycol, and liquid polyetheylene glycol, and the like), and suitable mixtures thereof. The proper fluidity may be maintained, for example, by the use of a coating such as lecithin, by the maintenance of the required particle size in the case of dispersion, and by the use of surfactants. Prevention of the action of microorganisms may be achieved by various antibacterial and antifungal agents, for example, parabens, chlorobutanol, phenol, ascorbic acid, thimerosal, and the like. In many cases, it may be preferable to include isotonic agents, for example, sugars, polyalcohols such as mannitol, sorbitol, or sodium chloride, in the composition. Prolonged absorption of the injectable compositions may be brought about by including in the composition an agent which delays absorption, for example, aluminum monostearate and gelatin.

Sterile injectable solutions may be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying, which yields a powder of the active ingredient plus any additional desired ingredient from a previously sterile-filtered solution thereof.

Oral compositions generally may include an inert diluent or an edible carrier. Oral compositions may be enclosed in gelatin capsules or compressed into tablets. For the purpose of oral therapeutic administration, the active compound may be incorporated with excipients and used in the form of tablets, troches, or capsules. Oral compositions may also be prepared using a fluid carrier for use as a mouthwash, wherein the compound in the fluid carrier is applied orally and swished and expectorated or swallowed. Pharmaceutically compatible binding agents and/or adjuvant materials may be included as part of the composition. The tablets, pills, capsules, troches, and the like, may contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. For administration by inhalation, the compounds are delivered in the form of an aerosol spray from a pressured container or dispenser which contains a suitable propellant, e.g., a gas such as carbon dioxide, or a nebulizer.

Agents or compositions described herein can also be used in combination with other therapeutic modalities, as described further below. Thus, in addition to the therapies described herein, one may also provide to the subject other therapies known to be efficacious for treatment of the disease, disorder, or condition.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intramuscular, intraperitoneal, intravenous, subcutaneous, intranasal, epidural, ophthalmic, buccal, or rectal administration.

### Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to stem cells, media, and factors as described herein. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents.

Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

The term “theranostic” refers to a portmanteau of therapeutics and diagnostics. A used herein “theranostic agent” refers to an “imaging agent”, a “therapeutic agent”, or a both.

As used herein “polypeptide” and “protein” are used interchangeably to refer to a polymer of amino acid residues.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art (see generally Remington's Pharmaceutical Sciences (A.R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Macrophages in Inflammatory Diseases and Malignancies

Macrophages are innate immune cells present in all major tissues and responsible for homeostasis. Macrophages sense and respond to pathogens and other environmental challenges and participate in tissue repair after injury. Recent research reveals macrophages as remarkably plastic cells that are epigenetically programmed in response to signals originating from the tissue environment. Macrophages integrate endocrine or paracrine signals with signals originating from phagocytosed cells, microvesicles, and molecules in the extracellular matrix. In addition, macrophages can interact directly with surface receptors on other tissue-resident cell populations, immune cells recruited during injury, and extracellular proteins. As a result, macrophages play diverse roles in the development, and the acute response to infection and tissue injury, tissue repair, as well as cancer development. Due to macrophages' tissue- and disease stage-specific roles, modulation of macrophage activity has been an active area in drug development for pharmacological intervention of inflammatory and malignant diseases. However, this proposes a new challenge for precise molecular phenotyping and targeting of macrophage subpopulations over the course of tissue injury and repair.

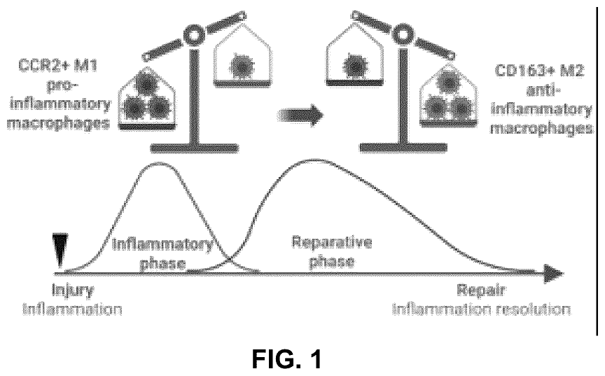

The macrophage heterogeneity and plasticity is evident from how the microenvironment shapes macrophage phenotype and functional identity, thus ensuring ongoing adaption of macrophages to the environment. Typically, macrophages are defined as M1 macrophages (classically activated pro-inflammatory macrophages) and M2 macrophages (alternatively activated tissue-resident anti-inflammatory macrophages). M1 macrophages are pro-inflammatory and have a central role in host defense against inflammation and infection, while M2 macrophages are associated with responses to anti-inflammatory reactions and tissue remodeling, and they represent two terminals of the full spectrum of macrophage activation (FIG. 1). Though the recent development of technologies enable the further categorization of tissue macrophage subsets based on the common life cycle properties and core gene signatures, the lack of consistent expression of characteristic surface biomarkers on certain macrophages across all major organs proposes challenges to determine their dynamic variation during the inflammatory process. Since the transformation of different phenotypes of macrophages regulates the initiation, development, and cessation of inflammatory diseases, it is critical to track the well-defined macrophage populations with constant expression of surface markers across multiple organs to interrogate their temporal-spatial distribution along the progression and regression of inflammatory diseases or malignancies. Due to the well-established biomarkers overexpressed on the cell surface and the dominant populations in the major organs, the conventionally defined M1 and M2 macrophages are still valuable to investigate their function and roles in the pathogenesis of inflammatory diseases.

Following the first phase of inflammatory response, C—C motif chemokine receptor 2 positive (CCR2+) blood monocytes derived from bone marrow are quickly recruited into the injured tissues, and then differentiated into CCR2+ pro-inflammatory macrophages to promote inflammation, which makes CCR2 a representative biomarker for M1 type macrophages. Of various biomarkers studied for macrophages, the scavenger receptor CD163 is a macrophage-specific protein and closely associated with the phenotype change of macrophages switching to alternative activated phenotypes upon inflammation such as repair mechanisms, as well as the progression of cancers. Although there is an increased interest in the soluble plasma CD163 that arises from the shedding of CD163 mediated by the tumor necrosis factor-α cleaving enzyme in a large spectrum of acute and chronic inflammatory disorders, the level of soluble CD163 is much lower than the membrane-bound form in macrophages, especially at inflammatory sites, which makes CD163 a unique target to determine dynamic variation of M2 macrophages in inflammatory diseases and malignancies.

### Molecular Imaging of Macrophages

In contrast to anatomy-based imaging modalities such as computed tomography (CT) or magnetic resonance, positron emission tomography (PET) provides unique advantages to sensitively and specifically detect the molecular signatures of macrophages subsets in vivo, as well as a well-established strategy for clinical translation. Many molecular probes have been developed to image surface biomarkers upregulated on macrophages in inflammatory diseases and malignancies in preclinical models, including somatastatin receptor subtype-2 (SSTR-2), mitochondrial membrane translocator protein (TSPO), mannose receptor or macrophage mannose receptor (MMR), C—X—C motif chemokine receptor 4 (CXCR4), myeloperoxidase (MPO), and metalloproteinases (MMPs). However, the overexpression of SSTR-2 on tumor cells may complicate the imaging of M1 type macrophages present in the tumor microenvironment, compounding investigations into their role in tumor progression or treatment response. For MMR imaging, a few tracers have been reported showing promising data detecting M2 type macrophages. However, the pharmacokinetics and targeting efficiency may need further improvement. For CXCR4, TSPO, and MPO, although many imaging agents have been developed for both preclinical and clinical studies, their expression on multiple cell types make these tracers less ideal for tracking specific type of macrophage.

[⁶⁴Cu]/[⁶⁸Ga]-DOTA-ECL1i radiotracers were developed to non-invasively quantify CCR2+ pro-inflammatory macrophages using PET in multiple pre-clinical animal models including atherosclerosis, cardiac injury, and lung injurylinflammation. The in vivo imaging specificity of these CCR2 tracers was demonstrated by a decreased signal in CCR2^{-/-} mice and near-complete loss of signal with competitive blockade. The imaging sensitivity was demonstrated in progressive and regressive animal atherosclerosis models. Importantly, the CCR2 tracer uptake correlated with the levels of macrophages at injured/diseased tissues, highlighting the potential of CCR2 as an imaging biomarker to determine M1 type pro-inflammatory macrophages in vivo. Moreover, the initial human studies in patients with pulmonary fibrosis demonstrated the importance of macrophage imaging to assess inflammatory diseases status and potential to guide molecular therapy. Indeed, the radiotracer is now involved in three other NIH funded human studies. To further improve the metabolic stability of the CCR2 tracer for more accurate detection of CCR2+M1 macrophages and expand its imaging research into neuroinflammation, new CCR2 targeted radiotracers was developed with more favorable imaging characteristics. Novel CD163 targeted PET tracer to detect M2 macrophages were also developed.

CCR2 targeted radiotracers imaging pro-inflammatory M1 macrophages were developed. The next generation CCR2 targeted radiotracers with improved imaging characteristics for CCR2 detection were developed, which include novel peptides screened from phage display for [⁶⁴Cu]/[⁶⁸Ga]/[⁸⁹Zr]/[¹⁸F] radiolabeling and small molecules for ¹⁸F labeling. The radiochemistry was optimized, the in vitro binding affinities, stability, pharmacokinetics, and in vivo imaging sensitivity and specificity were assessed. Novel radiotracers targeting CD163 to image resident macrophages were developed. CD163+ anti-inflammatory M2 macrophages play important roles in inflammation resolution and tumor progression. Although there is novel antibody developed for CD163, there is no reported small molecules or peptides binding CD163. Peptides were screened from phage display, first PET radiotracer targeting CD163 was developed, and a tool provided to determine its in vivo distribution and variation. The design was also optimized to have multiple versions of the radiotracers compatible with different radiolabeling strategies.

A pair of novel imaging agents to determine the dynamic temporal-spatial distribution of macrophage subsets in vivo to gain insight about their roles in the pathogenesis of inflammatory diseases and malignancies were provided. It is known that macrophage differentiation from monocytes occurs in the tissue in concomitance with the acquisition of a functional phenotype that depends on microenvironmental signals, thereby accounting for the many and apparently opposed macrophage functions, highlighting the importance to non-invasively track the dynamic distribution of macrophage subsets in vivo. Through this TR&D, a unique approach to measure the temporal-spatial distribution of CCR2+M1 pro-inflammatory was provided and CD163+M2 resident macrophages using the novel imaging agents developed. The structure of the tracers and radiolabeling strategies to enable imaging the spectrum of the inflammatory process was designed, thus gaining insight into the dynamic contribution of the two types of macrophages to the pathogenesis of inflammatory diseases and malignancies. Data regarding the performance of the radiotracers in their animal models was collected, which provided guidance to drive the further development and optimization CCR2 and CD163 targeted radiotracers. Importantly, the imaging of temporal-spatial distribution of M1 and M2 macrophages along the inflammation and malignancy were used to track their dynamic variation and provide guidance to design targeted molecular therapy or immunomodulatory treatment.

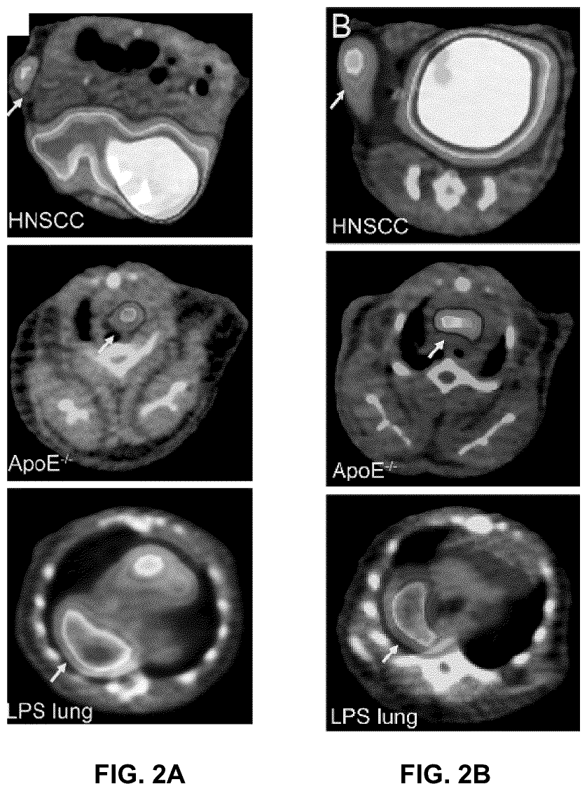

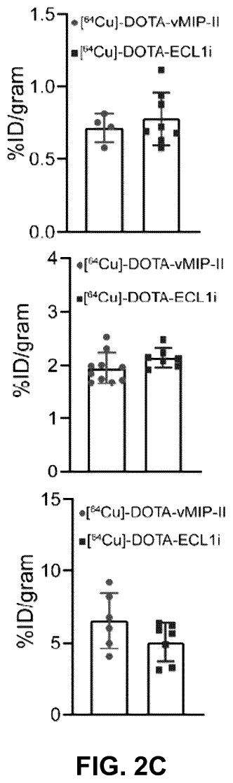

Assessment of ⁶⁴Cu-DOTA-vMIP-II Imaging chemokine receptors in inflammation and malignancy. The vMIP-II peptide is a sequence binding to 10 chemokine receptors with high affinities, which makes it unique for exploring cellular inflammation mediated by a group of chemokine receptors in animal models. As show in FIG. 2A, [⁶⁴Cu]-DOTA-vMIP-II PET showed intensive uptake in the HNSCC tumor, atherosclerotic lesion, and lung inflammation induced by lipopolysaccharide (LPS), indicating the potential of this radiotracer for inflammation imaging. [⁶⁴Cu]-DOTA-ECL1i revealed comparable imaging efficiency to the broad-spectrum [⁶⁴Cu]-DOTA-vMIP-II tracer (FIG. 2B and FIG. 2C).

Translation of [⁶⁴Cu]-DOTA-ECL1i for human Imaging. Animal toxicity studies with non-radioactive Cu-DOTA-ECL1i, dosimetry using [⁶⁴Cu]-DOTA-ECL1i in naïve mice were performed and submitted the exploratory investigative new drug (eIND) application to US Food Drug Administration (FDA). Following approvals from FDA, Washington University Radioactive Drug Research Committee (RDRC) and Institutional Review Board (IRB), the human dosimetry studied in six healthy volunteers (3 males, 3 females) who were injected with [⁶⁴Cu]-DOTA-ECL1i (5.2 mCi-10.4 mCi) and scanned at 15-30 min, 2-4 h, 20 h, and 44 h post injection was initiated. All patients showed rapid distribution of the tracer and fast clearance through the urinary tract. Approximately 80% of the activity was excreted in the urine within 2-4 hours after injection. The most intense activity was seen in the liver and kidneys, while the majority of tracer was excreted in urine. The gender average Effective Dose was 0.064±0.012 rem/mCi (0.073 rem/mCi for females and 0.056 rem/mCi for the males). The critical organ for both genders was the urinary bladder wall, with the highest average estimated dose derived from the female model at 0.45 rad/mCi. Therefore, a dose of 8-10 mCi was injected to each patient in the HNSCC patient studies.

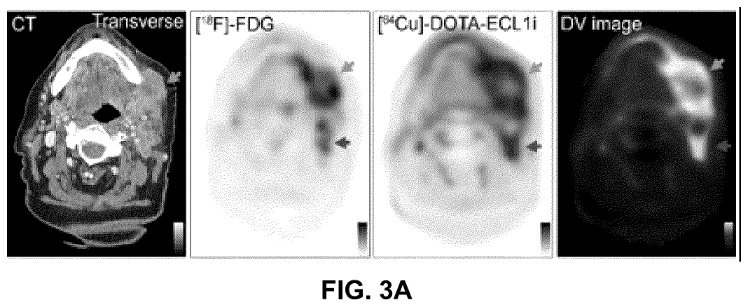

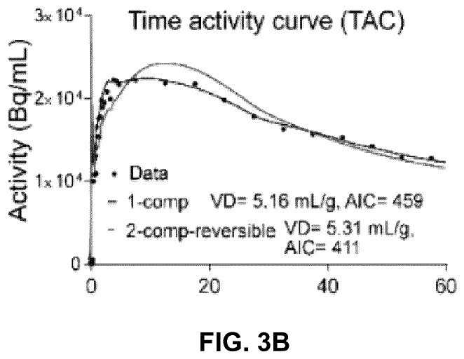

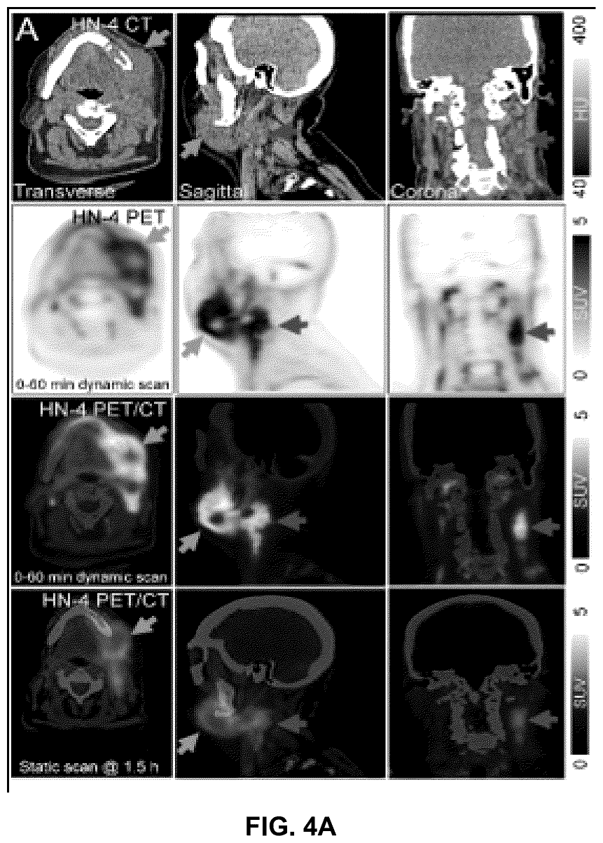

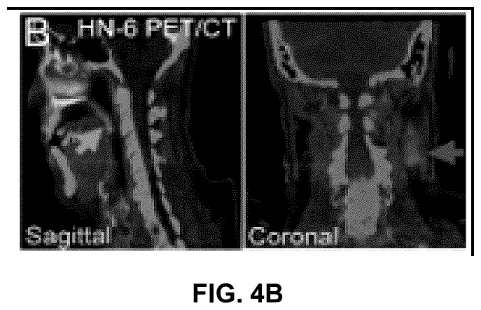

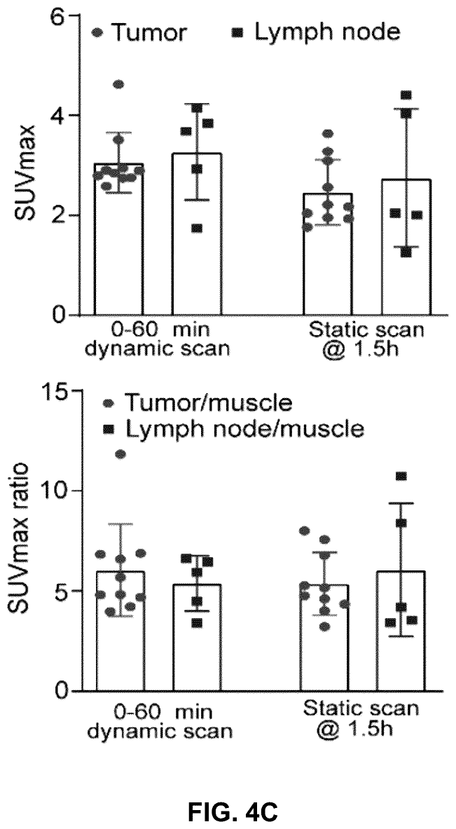

CCR2 PET Imaging in HNSCC patients. Due to the dramatic negative effect of the COVID-19 pandemic on the performance of clinical research, HNSCC patient imaging study was significantly impacted. Although numerous strategies that integrate the efforts of the TR&D, Head and Neck Surgery, Nuclear Medicine and the MIR Research Facilities to enhance recruitment and study performance, participation from HNSCC patients was low. 10 HNSCC patients were imaged and tissues were collected from 9 of them. The PET imaging in these patients showed significant tracer uptake in tumors and lymph nodes. Among them, patient HN-4 was a 72-year-old female with pT4aN3bM0 oromandibular poorly-differentiated keratinizing SCC, multiple lymph node metastases, and extra nodal extension. Clinical CT identified the primary tumor (green arrow) and lymph node (LN) (red arrow) metastasis (FIG. 3). The CCR2 radiotracer [⁶⁴Cu]-DOTA-ECL1i signal was intense and in close proximity to the uptake of [¹⁸F]-FDG-PET/CT study. Inspection of CCR2 tracer tumor time activity curves showed reversible accumulation. Kinetic modeling by the Quantitative Imaging and Informatics Resource (Q12R) showed the reversible 2-compartment model (4 parameters) fitted better than 1-compartment model (confirmed by akaike information criteria (AIC)). Furthermore, parametric Logan images provided regional Distribution Volume (DV), which would be compared to CCR2 immunostaining to validate imaging efficiency. In contrast to [¹⁸F]-FDG, the different uptake pattern of [⁶⁴Cu]-DOTA-ECL1i may be reflective of TAMs within tumor immune microenvironment. Moreover, representative CCR2 PET/CT images of HN-4 (FIG. 4A) and HN-6 (p16+) (FIG. 4B) showed that [⁶⁴Cu]-DOTA-ECL1i specifically detected the primary tumors (green arrow) and LN (red arrow) metastasis. Quantitative uptake analysis demonstrated significant uptake for primary tumor and LN during the dynamic and at 1.5 h post injection images. Moreover, the primary tumor and LN uptake was stable in-between the two scans, suggesting radiotracer binding and retention (FIG. 4C). The target-to-background ratios demonstrated an appropriate contrast between the tumor and the surrounding non-malignant tissue for accurate detection of primary tumors and LN metastasis.

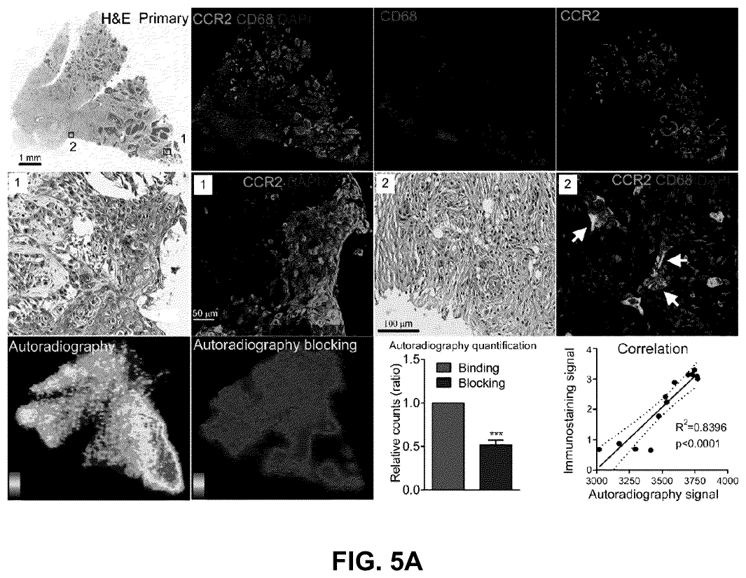

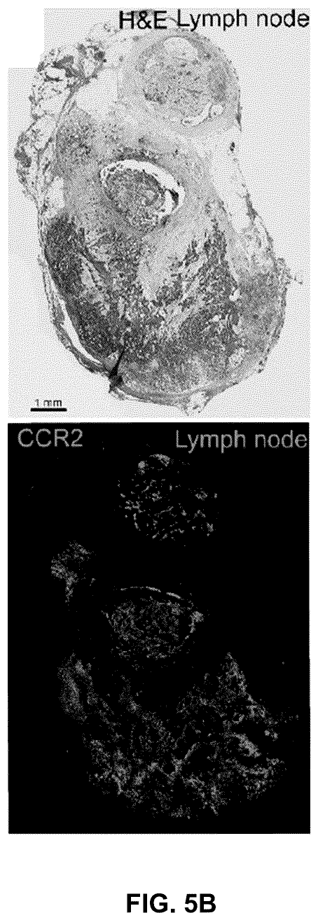

Ex vivo HNSCC tissue characterization. In the six HNSCC patients, immunostaining showed strong expression of CCR2 on tumor cells and infiltrated TAMs in all primary tumors (n=9) and LNs (n=5), while the degree of CCR2 and CD68 co-localization varied among these specimens. For example, histology (H&E) of the collected primary tumor specimen from HN-4 showed that the tumor is a well-differentiated keratinizing SCC with a significant amount of infiltrative mononuclear inflammatory cells (FIG. 5A). Immunofluorescence staining showed intensive expression of CCR2+ cells and CD68+ TAMs with a significant number of cells positive for both (yellow). Ex vivo tumor autoradiography revealed strong [⁶⁴Cu]-DOTA-ECL1i binding and the specificity was confirmed by the competitive receptor blocking and correlation analysis. Moreover, the lymph node with metastatic carcinoma collected from this patient also showed strong CCR2 staining (FIG. 5B), which was consistent with the in vivo CCR2 tracer uptake (FIG. 4).

Human blood metabolism of [⁶⁴Cu]-DOTA-ECL1i and optimization of tracer design. Venous blood metabolism assay for every HNSCC patients at 60 min post injection was performed. The radiotracer showed variable stability from 25.1% to 100% (62.8±25.9%, n=7), which highlighted the importance to improve the radiotracer stability for more accurate detection of CCR2 in humans. The design of ECL1i tracer was optimized following several strategies including 1) using other macrocyclic chelators including NOTA and NODAGA; 2) cyclization of the ECL1i peptide, 3) conjugation of chelators via the N-terminus using amide bond instead of the maleimide-thiol conjugation via the cysteine residue at the C-terminus. In total, 6 new tracers were synthesized for comparison. In vitro binding assays using THP-1 cells showed high binding affinities (IC₅₀=30.8-40.0 nM), which were comparable to that of [⁶⁴Cu]-DOTA-ECL1i (IC₅₀=58.3±17.7 nM). Importantly, these new tracers (i.e. [⁶⁴Cu]-NODAGA-ECL1i) demonstrated improved in vivo stability compared to [⁶⁴Cu]-DOTA-ECL1i, and comparable or slightly better targeting efficiencies (tumor uptake, tumor/muscle ratios) to that of [⁶⁴Cu]-DOTA-ECL1i. Therefore, the TAC and EAC recommended thorough tracer stability evaluation such as microsomal assay or non-human primate (NHP) to assess the metabolic stability for future tracer development.

Taken together, [⁶⁴Cu]-DOTA-ECL1i for CCR2 imaging in HNSCC patients has been successfully translated. CCR2 PET revealed the sensitive detection of the tumors and lymph nodes and the ex vivo characterization of the tissues collected from these patients demonstrated the overexpression of CCR2 positive pro-inflammatory M1 macrophages in tumor microenvironment and the potential of CCR2 as a molecular biomarker for HNSCC prognosis. The ongoing CCR2 clinical imaging studies in other patient populations all show very encouraging preliminary data. These findings are the key motivations to drive us to further develop CCR2 targeted tracers with improved stability, enhanced targeting efficiency, and different physicochemical properties (i.e. small molecules) to expand the CCR2 imaging of inflammatory diseases. Novel CD163 targeted PET tracer imaging M2 anti-inflammatory macrophages were also developed and to non-invasively track the dynamic variation of these two types of macrophages in vivo to better understand the temporal-spatial distribution of these cells and related function/contribution to the pathogenesis of inflammatory diseases, their resolution, and malignancies. The approach to develop the Novel CD163 targeted PET tracer imaging M2 antiinformmatory macrophages are below.

### Develop New CCR2 Targeted Radiotracers Imaging M1 Pro-Inflammatory Macrophages.

Rational and study design. New CCR2 tracers to improve the imaging efficiency determining CCR2+M1 macrophages and extend the scope of CCR2 imaging were developed. Based on the specific imaging request and animal models used, the structure of the precursor was designed and the radiotracer synthesized with appropriate radiolabeling strategies. The CCR2 tracer stability, in vitro binding affinities, pharmacokinetics, and in vivo targeting efficiencies following pre-designed benchmarks of success were assesssed. Radiotracers/precursors were provided for in vivo PET imaging in appropriate biological systems.

Small molecule based CCR2 radiotracer labeled with ¹⁸F were synthesized for neuroinflammation imaging. An ¹⁸F-labeled CCR2 radiotracer with desired characteristics such as the appropriate logP/lipophilicity to cross the blood-brain-barrier (BBB), radiolabeling yield, and stability was synthesized. The upregulation of CCR2 in multiple sclerosis is well documented. In mouse experimental autoimmune encephalomyelitis (EAE) model, the BBB is compromised at later stage. The peptide-based CCR2 tracers with ¹⁸F-labeled small molecules for MS imaging at various stages were compared. The design of these CCR2 tracers were improved to balance targeting effect vs. BBB penetration. To further improve the tracer stability and minimize metabolites in vivo, the CCR2 peptide-NODAGA conjugate for ⁶⁴Cu and ⁶⁸Ga radiolabeling were synthesized. The radiolabeling specific activities, stabilities, binding affinities, and imaging efficiencies to address the needs for chronic and acute animal heart injury models were compared. Nanobody based radiotracer was also developed. A proof-of-concept study was performed using ^{99m}Tc labeling through the histidine-tag and a conjugation strategy was designed to synthesize ⁶⁴Cu labeled PET tracer. In addition to the DOTA/NODAGA chelation strategies, a heptadentate aminopolycarboxylate ligand with a 1,4-diazepine scaffold (AAZTA) conjugated precursor specifically was synthesized for trivalent metal with a coordination number of 8, such as scandium-44 (⁴⁴Sn). Moreover, the peptide-DOTA conjugate can also be used for ⁸Zr labeling to study the longitudinal distribution and uptake in tumors. Since the localization of abdominal aortic aneurysm is close to the kidneys, a ⁶⁸Ga radiolabel was used due to its faster blood and renal clearance than ⁶⁴Cu labeled counterpart. The chelator was changed to alter the charge of the tracer or design different conjugation strategy to optimize the charge distribution of the tracer to facilitate the clearance for optimal target-to-background ratio. Besides the optimization of chelators, the pharmacokinetics of ⁶⁴Cu labeled CCR2 peptide tracer was improved by introducing polyethylene glycol (PEG) to extend the blood circulation for increased tumor uptake.

Sex as a biological variable. Inflammatory diseases and cancers happen to both men and women. Thus, both male and female mice were used throughout the proposed studies. However, the sample size was too small to formally evaluate the impact of this variable on the measurement.

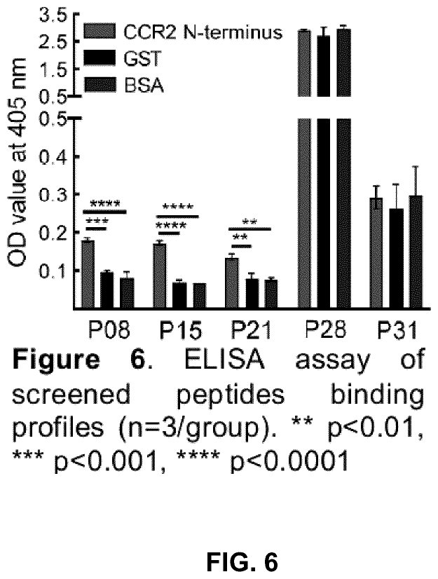

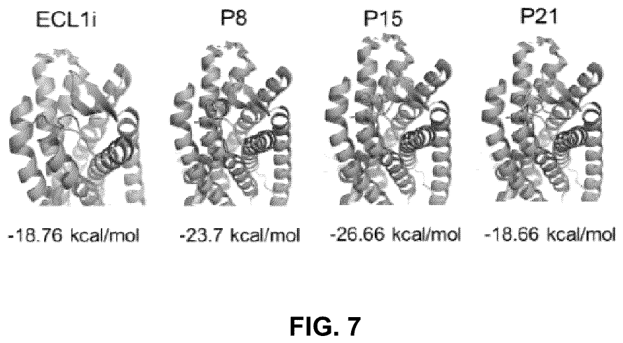

Phage display screening and identification of new CCR2 binding peptides. Using a recombinant human CCR2 N-terminus protein (1-42 amino acids) with a glutathione s-transferase (GST) tag, phage display screening was performed using Ph.D.™-12 peptide library to identify sequences binding to CCR2. After three rounds of phage selections, 50 phages were selected to amplify in E. coli, titer, and for DNA sequencing. Enzyme-linked immunosorbent assay (ELISA) was performed with pre-selected phages and identified five peptide sequences showing potential binding to CCR2. These peptides conjugated with biotin labels were customized for further peptide ELISA assays. As shown in FIG. 6, 3 peptides (P08 (VDDHYGTIVRNG, SEQ ID NO: 2), P15 (HWKWLKPWAVNT, SEQ ID NO: 3), and P21 (LIPAPGGTPRAG, SEQ ID NO: 4), all at the concentration of 104 M) out of the 5 sequences showed significantly higher binding to CCR2 N-terminus protein compared to the GST tag alone and bovine serum albumin (BSA, negative protein control). Therefore, these peptides were selected as CCR2 binding sequences to synthesize peptide-chelator conjugates. To further confirm the specific binding of these peptides, peptide docking studies were performed based on a crystal structure of CCR2 (PDB Id 6GPS) and receptor structures resulting from molecular dynamics simulations of CCR2 and endogenous ligand CCL2 complex using the program AutoDock CrankPep. As shown in FIG. 7, consistent with ECL1i peptide, P8, P15 and P21 all bind to the same domain (extracellular loop 1, homologous between human and mouse) of CCR2 while P15 has the best binding affinity (−26.66 kcal/mol) among the three sequences.

Summary of preliminary data. Phage display screening of CCR2 binding peptides was performed and 3 candidate sequences were identified. Molecular modeling demonstrated that these peptides had comparable or superior binding affinities comparing to ECL1i, suggesting the potential of these peptides for further evaluation.

### Screen and Optimize New Ligands Including Peptides and Small Molecules Targeting CCR2

Synthesis, radiolabeling and assessment of stability of new peptide-based CCR2 PET tracers. The P15 peptide with protecting groups was customized and NODAGA-NHS or DOTA-NHS esters conjugated to the N-terminus of the sequence through the histidine residue. The protection groups were removed and the NODAGA/DOTA-P15 conjugate was purified using high performance liquid chromatography (HPLC). The conjugate was characterized with HPLC-mass spectrometry (HPLC-MS) prior to radiolabeling with ⁶⁴Cu or ⁶⁸Ga following well-established radiolabeling protocols. The [⁶⁴Cu]/[⁶⁸Ga]-NODAGA/DOTA-P15 tracer was subjected to multiple levels of stability assays in vitro including 1) radiolabel stability using ethylenediaminetetraacetic acid (EDTA) challenge in buffer; 2) serum stability in mouse/human serum after 1 h incubation; and 3) metabolic stability assay using human liver microsomes following commercially available protocols.

Benchmark of success: NODAGA/DOTA-P15 conjugate purity (≥95%), [⁶⁴Cu]/[⁶⁸Ga]-NODAGA/DOTA-P15 radiochemical purity (≥95%), radiolabeling specific activity (≥1.5 mCi/nmol), radiolabel stability (≥95%), serum stability (≥95%), and metabolic stability (≥95%).

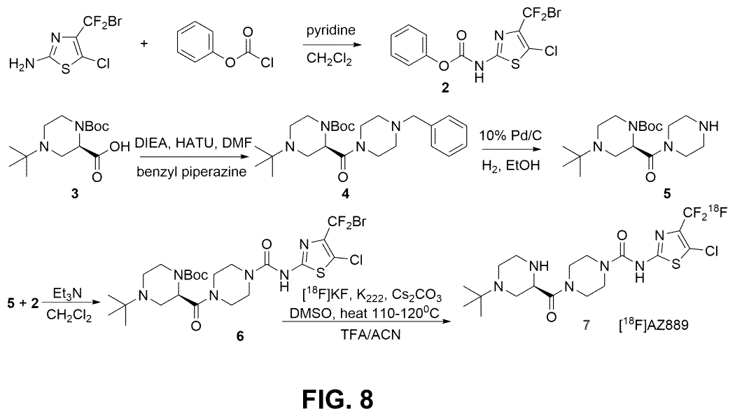

Synthesis, radiolabeling and assessment of stability of small molecules-based CCR2 PET tracers. Of many CCR2 antagonists, AZ889 is of particular interest due to its high binding potency (IC₅₀=0.13 nM) to CCR2 and potential for ¹⁸F radiolabeling through the CF₃ group. Therefore, the precursor 6 according to the scheme outlined in FIG. 8 was synthesized. Radiolabeling with ¹⁸F was done through the nucleophilic substitution, a well-established strategy for synthesizing [¹⁸F]CF₂-group. Following HPLC purification, [¹⁸F]AZ889 was formulated with 10% ethanol in saline for assessments outlined above.

Benchmark of success: precursor 6 purity (≥95%), [¹⁸F]AZ889 radiochemical purity (≥95%), radiolabeling specific activity (≥1.0 mCi/nmol), serum stability (≥90%), and metabolic stability (≥90%).

### Assess the Binding Affinities, Pharmacokinetics and In Vivo Imaging Efficiency of New CCR2 Tracers

In vitro cell binding assay. The binding affinities of all CCR2 radiotracers were determined using a CCR2-expressing human monocyte cell line THP-1 while CCR2 negative 293T cells were used as controls. The IC₅₀ values of these radiotracers were determined using a competitive receptor binding assay with non-radioactive counterpart at room temperature at various concentrations and compare with the current tracer [⁶⁴Cu]-DOTA-ECL1i (IC₅₀=58.3±17.7 nM).

In vivo pharmacokinetics. The pharmacokinetic studies of these radiotracers were carried out in both male and female wild type C57BL/6 mice following well-established protocols. For ⁶⁴Cu radiolabeled P15 peptide radiotracers, biodistribution was performed at 1 h, 4 h, and 24 h (n=5/group) post intravenous injection via tail vein. For ⁶⁸Ga labeled CCR2 tracers, the time points were 1 h, 2 h, and 4 h (n=5/group). For [¹⁸F]AZ889, the organ collections were performed at 1 h, 4 h and 6 h (n=5/group) post injection. Particularly, the brain accumulation of this radiotracer was measured at 5 min post injection in rat to determine the blood-brain-barrier (BBB) penetration of [¹⁸F]AZ889 following a defined guidline.

In vivo targeting efficiency. The CCR2 targeting efficiencies of the as-developed radiotracers was assessed in a mouse atherosclerosis model using apolipoprotein E knockout (ApoE⁴) mice (both male and female, n=5/group) fed on high fat diet (HFD) that is a chronic inflammatory model known with high CCR2 expression at atherosclerotic plaques. The chronic nature of this model will enable the cross comparison of the targeting effect of different radiotracers within reasonable timeframe while the inflammatory status and CCR2 expression is less variable. For PET imaging, 0-60 min dynamic scan will be performed on all the mice. All PET images generated from this TR&D and those retrieved from CPs will be collectively analyzed by Q12R and TR&D2. Partial volume effect will be corrected to accurately determine the tracer uptake. Targeting effect was assessed by the SUV uptake at the aortic arch in ApoE^{-/-} mice at least 10 weeks post HFD, at time point in which the upregulation of CCR2 was documented. The imaging specificity of various CCR2 radiotracers was assessed via competitive receptor blocking studies using non-radioactive counterparts in excess amount (100-500 fold). The significant decrease of tracer uptake indicated the CCR2 targeting specificity. The imaging sensitivity assessment for each CCR2 radiotracer was carried out by scanning the same cohort of ApoE^{-/-} mice along the progression of the disease at 10, 16 and 24 weeks post HFD. Separate cohorts of ApoE^{-/-} mice was used for aortic artery tissues characterization including real-time polymerase chain reaction (RT-PCR) assay, immunofluorescent staining, and flow cytometry to comprehensively assess the expression of CCR2 at the corresponding time points following the reported protocols. PET tracer uptake at aortic arch was integrated with these CCR2 tissue characterizations to assess the imaging sensitivity of these radiotracers detecting CCR2 (please see vertebrate animals section).

In vivo metabolism and PET Imaging in non-human-primate (NHP). Radiotracers that performed well in mouse studies were further assessed their metabolic stabilities using NHP and to assess the BBB penetration for [¹⁸F]AZ889 following well established procedures. Arterial blood samples from NHPs were collected at multiple time points post intravenous injection and plasmas were separated, filtered, and injected into radioactive HPLC to determine the radioactive metabolites. A 0-60 min dynamic PET scan in NHP with candidate CCR2 radiotracers was performed and the tracer accumulation in the whole-body and particularly brain for small molecule radiotracers was quantified. The time-activity-curve of tracer retention in brain with the blood curve obtained using gamma counting was compared to calculate the brain-to-blood ratio to assess the BBB penetration.

Benchmark of success: IC₅₀ (<10 nM), excretion (>80% injected dose at 4 h), brain retention (small molecule CCR2 tracer in rat only: >0.1% ID/g, >0.01% ID/g in NHP), in vivo stability in NHP (≥80% at 1 h), metabolic stability (≥90% at 1 h), targeting specificity (≥50% blocking effect), target-to-background ratio (Z 5).

Data/Statistical analysis. Descriptive statistics were used to summarize imaging, immune activity and pathological properties of the murine atherosclerosis model. The tracer uptake was correlated with CCR2+ cells burden using a Spearman's correlation coefficient. Differences in the characteristics (disease severity, disorganization, presence of CCR2) between the atherosclerotic lesions of the aortic arch specimens was evaluated using chi square tests. Differences in tracer uptake between the new CCR2 tracers and current ⁶⁴Cu-DOTA-ECL1i were assessed using a Mann-Whitney U test. Due to small sample size, all statistical test were based on the exact p value methods. Power calculation. The correlation between CCR2 tracer uptake and CCR2+ were very high at 0.935, and 5 mice were used, which achieved 80% power to detect a correlation of 0.935 using a two-sided test with 5% significance level.

### Develop CD163 Targeted Radiotracer Imaging M2 Tissue Resident Macrophages.

Rational and study design. A novel CD163 targeting PET tracers was developed and the stability, binding affinity, imaging sensitivity and specificity to M2 tissue resident macrophages were assessed.

### Screening and Synthesis of CD163 Binding PET Radiotracer.

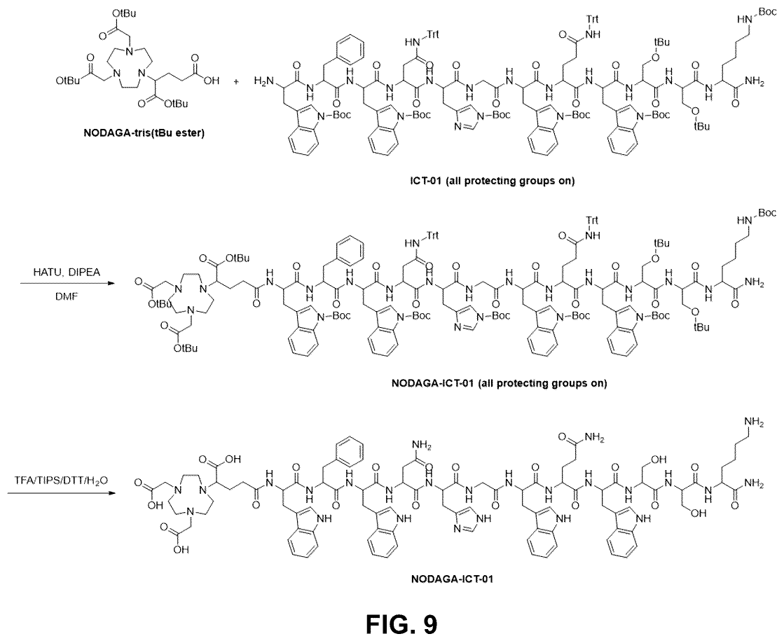

Phage display screening of CD163 binding peptides using the Ph.D.-12™ library and CD163 protein was performed. After three rounds of phage selection, phage ELISA, and peptide ELISA, five peptide sequences binding CD163 were identified. The peptide sequences were customized for PET tracer synthesis, and identified one leading sequence names ICT-01 WNHGWFWQWSSK (ICT-01 SEQ ID NO: 1) as a candidate for radiotracer development. As shown in FIG. 9, NODAGA chelator was conjugated to the N-terminus of ICT-01 (NODAGA-ICT-01) via an amide bond for radiolabeling with ⁶⁴Cu showing high specific activity (2 mCi/nmol), which enabled trace amount administration for PET imaging. Initial mouse serum stability studies showed that the ⁶⁴Cu-NODAGA-ICT-01 was 100% stable after 1 h incubation.

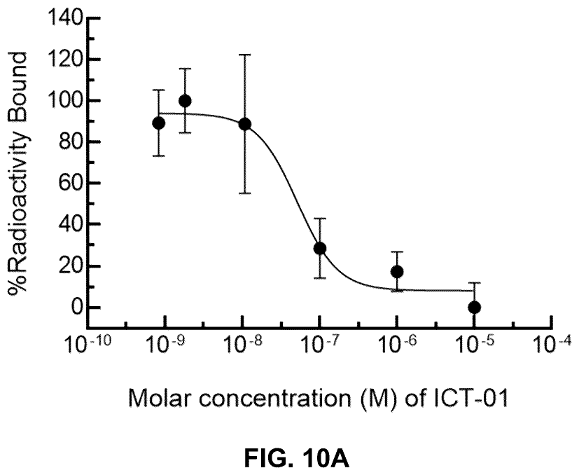

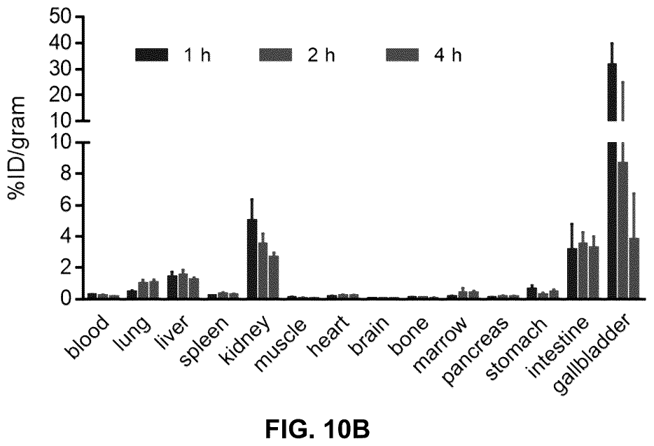

In vitro cell binding assay of [⁶⁴Cu]-NODAGA-4CT-01. The in vitro cell binding assay of [⁶⁴Cu]-NODAGA-ICT-01 was performed in CD163 overexpressing U87 cells. As shown in FIG. 10A, [⁶⁴Cu]-NODAGA-ICT-01 demonstrated high binding affinity to CD163 with an IC₅₀ of 26.8 t 21.4 nM (n=3).

Biodistribution of [⁶⁴Cu]-NODAGA-4CT-01. In vivo pharmacokinetics of [⁶⁴Cu]-NODAGA-ICT-01 was performed in wild type C57BL/6 mice at 1 h, 2 h, and 4 h post injection via tail vein. As shown in FIG. 10B, [⁶⁴Cu]-NODAGA-ICT-01 showed effective renal clearance and low retention in most organs. The blood pool organs (blood, heart, lung) had less than 0.5% ID/gram retention at 1 h post injection while the liver uptake was less than 2% ID/gram at all times. Interestingly, gallbladder showed high retention of [⁶⁴Cu]-NODAGA-ICT-01 (31.8±8.1% ID/gram) at 1 h, followed by rapid decrease to less than 4% ID/gram at 4 h, which still ensured acceptable dosimetry for potential clinical translation based on preliminary estimation.

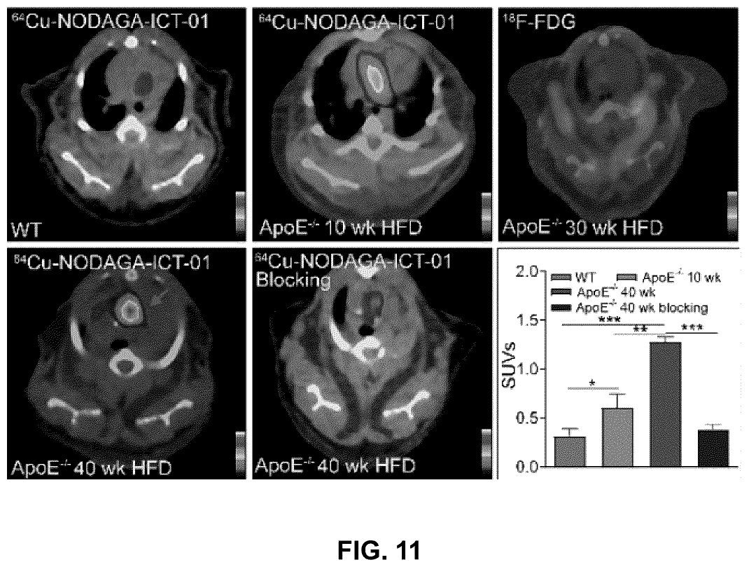

CD163 PET imaging atherosclerosis. [⁶⁴Cu]-NODAGA-ICT-01 imaging of CD163 was assessed in an ApoE^{-/-} mouse atherosclerosis model. As shown in FIG. 11, consistent with the rapid blood clearance determined in the biodistribution study shown in FIG. 10, low tracer uptake was observed at aortic arch in WT mice. In contrast to the low accumulation of [¹⁸F]-FDG in ApoE^{-/-} mice at 30 weeks post HFD, a strong [⁶⁴Cu]-NODAGA-ICT-01 signal was determined at aortic arch in ApoE^{-/-} mice after 10 week HFD, which was doubled at 40 weeks, consistent with the increased M2 resident macrophages in atherosclerosis. The competitive blocking study revealed approximately 3-fold decrease of tracer uptake, suggesting the targeting specificity.

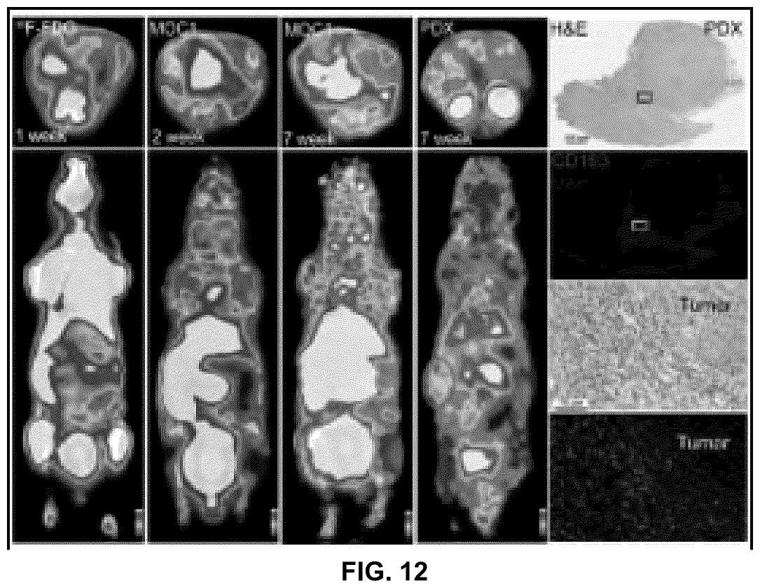

CD163 PET imaging tumors. Preliminary CD163 imaging in mouse HNSCC tumor models was also performed. As shown in FIG. 12, [¹⁸F]-FDG PET revealed tumor ^{localization at} 1 week post implantation of MOC1 tumor cells. [⁶⁴Cu]-NODAGA-ICT-01 showed intensive tumor uptake at 2 weeks in the same tumor-bearing mouse, which significantly increased at 7 weeks post implantation, indicating the sensitivity of CD163 PET. Moreover, in HNSCC patient derived xenograft (PDX) mouse model, [⁶⁴Cu]-NODAGA-ICT-01 also demonstrated the sensitive detection of tumor. Ex vivo tissue histology showed the infiltration of immune cells that were largely positive for CD163 while the tumor cells were mostly negative, supporting the PET data.

Summary of preliminary data. One candidate tracer [⁶⁴Cu]-NODAGA-ICT-01 for CD163 PET imaging was identified and synthesized. Initial evaluations demonstrated the stability of the radiotracer in mouse serum and in vitro cell binding studies showed high binding affinity to CD163. Biodistribution showed fast renal clearance and low retention in major organs in WT mice. PET imaging in ApoE^{-/-} mice atherosclerosis model and HNSCC models demonstrated the potential of [⁶⁴Cu]-NODAGA-ICT-01 for further evaluation in inflammatory animal models.

### Screen and Synthesize Novel Peptide Based Tracers Binding CD163.

Preparation, optimization, and assessment of new peptide-based CD163 PET tracers. The initial evaluation of [⁶⁴Cu]-NODAGA-ICT-01 demonstrated the potential of this radiotracer targeting CD163 in vivo. In vitro evaluation to assess the metabolic stability of this radiotracer using the microsomal assay was performed. The DOTA and AAZTA chelator conjugated ICT-01 for various radiometals (i.e. ⁴⁴Sc, ⁸⁹Zr) labeling was synthesized. The pharmacokinetics of CD163 tracer was optimized by conjugating a bifunctional PEG linker (i.e. PEG4) in between the chelator and ICT-01 peptide to extend the blood circulation for improved targeting in vivo. The in vitro binding assays using U87 cells were carried out to screen those tracers with desirable binding affinities.

Biodistribution will be carried out in WT mice to assess the distribution and retention in major organs.

Benchmark of success: Chelator-ICT-01 conjugate purity (≥95%), ⁶⁴Cu/[⁶⁸Ga]-chelator-ICT-01 radiochemical purity (≥95%), radiolabeling specific activity (21.5 mCi/nmol), radiolabel stability (≥95%), serum stability (≥95%), metabolic stability (≥95%), IC₅₀ (<10 nM), excretion (>80% injected dose at 4 h).

### Assess CD163 PET Tracer Imaging Sensitivity, Specificity and Targeting Efficiency In Vivo.

The candidate lead CD163 radiotracers were subject to NHP study to assess their in vivo stability and pharmacokinetics following the protocols outlined above. The in vivo targeting efficiency of candidate CD163 PET tracers were first assessed in ApoE^{-/-} mouse atherosclerosis model due to its suitability to cross compare multiple tracers. The imaging sensitivity and specificity were assessed following the approaches outlined above. Aortic arteries from the ApoE^{-/-} mice were collected for multiple characterizations of CD163 expression including flow cytometry, immunostaining, and RT-PCR at the corresponding time points of PET imaging, which were integrated with PET data to assess the targeting efficiency of the various CD163 tracers.

Benchmark of success: In vivo stability in NHP (≥80% at 1 h), targeting specificity (>50% blocking effect), target-to-background ratio (Z 5).

Data/Statistical analysis. Descriptive statistics were used to summarize imaging, immune activity and pathological properties of the murine atherosclerosis model. The tracer uptake were correlated with CD163 cells burden using a Spearman's correlation coefficient. Differences in the characteristics (disease severity, disorganization, presence of CD163) between the atherosclerotic lesions of the aortic arch specimens were evaluated using chi square tests. Due to small sample size, all statistical test were based on the exact p value methods. Power calculation. The correlation between C163 tracer uptake and CD163 cells burden were very high at 0.935, and 5 mice were use, which achieve 80% power to detect a correlation of 0.935 using a two-sided test with 5% significance level.

### Validate Macrophage-Specific Radiotracers to Detect Heterogeneity, Temporal-Spatial Distribution of M1 and M2 Macrophages in Inflammatory Diseases and Malignancies.

Rational and study design. CCR2+M1 and CD163+M2 macrophages are dynamically expressed and temporally localized in inflammatory diseases and malignancies. The CCR2 and CD163 PET signals were interrogated to localize the temporal-spatial distribution of M1 and M2 macrophage subtypes in vivo and validate their relationship in animal inflammation and tumor models.

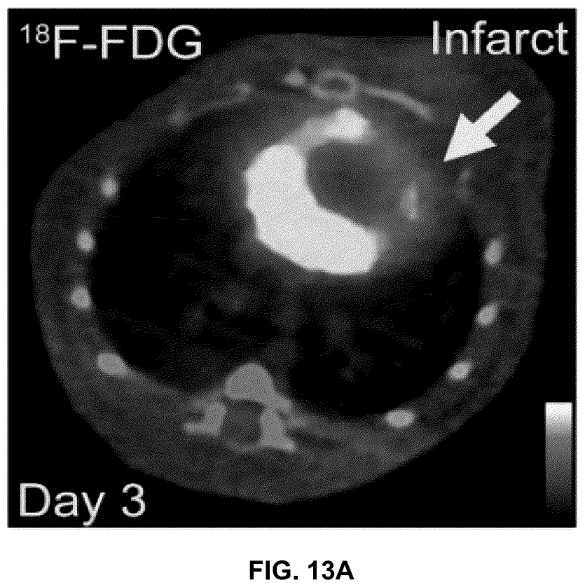

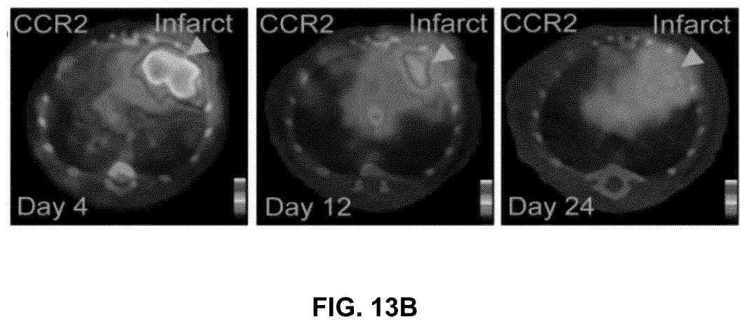

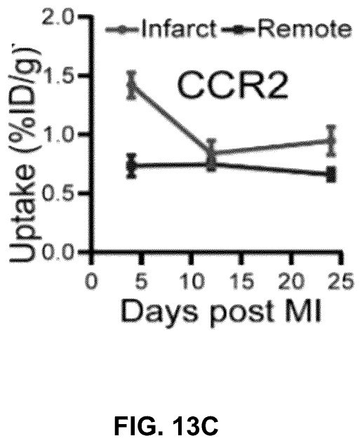

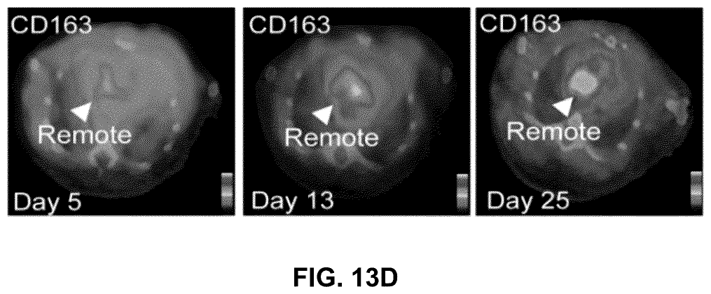

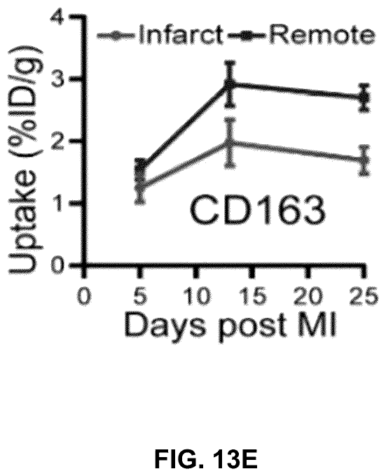

Preliminary data. Based on the currently available radiotracers, [⁶⁸Ga]-DOTA-ECL1i and [⁶⁸Ga]-NODAGA-ICT-01 PET imaging in an ischemia reperfusion injury induced myocardial infarction mouse model was performed to determine the temporal-spatial distribution of M1 CCR2+ pro-inflammatory and M2 CD163+ tissue resident macrophages post MI and during the remodeling process. The rapid decay of ⁶⁸Ga (t/2=68 min) enabled PET imaging in MI mice in two consecutive days with [⁶⁸Ga]-DOTA-ECL1i and ⁶⁸Ga-NODAGA-ICT-01. As shown in FIG. 13A, [¹⁸F]-FDG identified the infarct at day 3 post MI. Day 4 CCR2 PET revealed intensive tracer uptake at infarct zone, which gradually decreased through the remodeling process while the tracer accumulation at remote area remained stable during the study (FIG. 13B, C). Interestingly, CD163 PET signals were observed at remote area and gradually increased from day 5 to day 13 and then remained stable until day 25 (FIG. 13D, E). Though the role of tissue resident macrophages in chronically failing heart is well documented, little is known about their temporal-spatial distribution post MI. The data revealed the dynamic change of these two types of macrophages for the first time, which really highlighted the importance of PET imaging as a non-invasive tool to track macrophage subsets in vivo to gain more insight into their role in the injury and remodeling process.

Summary of preliminary data. The feasibility of using PET imaging to detect the dynamic variation of macrophage subsets in vivo was demonstrated. The information will be important to understand the roles of M1 and M2 macrophages in the acute and the chronic phase post MI.

### Determine the M1 CCR2+ and M2 CD163+ Macrophage Subtypes, Temporal-Spatial Distribution in Inflammation and Malignancies.

PET imaging of macrophage subsets dynamics in animal inflammatory models. The newly developed CCR2 and CD163 tracers were used to determine the dynamic change of M1 and M2 macrophage subsets. For animal models with acute inflammation such as MI, ¹⁸F or ⁶⁸Ga as radiolabels for CCR2 or CD163 tracers were used due to their rapid decay and clearance. This enabled serial PET imaging within a short time to capture the rapid change of these two types of macrophages along the promotion of inflammation and inflammation resolution phases. For animal models with chronic inflammation such as atherosclerosis or PDX mice, ⁶⁴Cu radiolabeled tracers were appropriate for serial imaging due to the suitable half-life, image resolution, and relatively slow change of inflammatory cells within a short time (i.e. one week). Tracer uptake were quantified at each time points to cross compare the relative change overtime to gain insight of the temporal-spatial distribution of M1 CCR2+ and M2 CD163+ macrophages during the initiation and progression of diseases.

Integrate the Expression of CCR2 and CD163 with Corresponding PET Signals to Correlate with the Status of Diseases

Histopathological characterization and immune cell profiling of inflammatory tissues. At each imaging time point, tissues were collected for multiple histopathological characterization including histology, RT-PCR, immunostaining and flow cytometry (n=10/group). H&E and pentachrome staining were performed to assess the severity of disease and infiltration of inflammatory cells. Immunofluorescent staining were carried out to stain CCR2, CD68, and CD163, as well as p63 for tumor cells in malignant tissues to investigate the heterogeneous distribution of the two macrophage populations. Autoradiography for each tracer in individual models was performed to examine the intra-tissue distribution of tracer binding and correlate with immunostaining profiles to confirm binding specificity. Freshly collected tissues were digested to a single cell suspension. For CCR2, cells were stained with antibodies specific for CD45, CD64, Ly6C, and MHC-II. GFP reporter florescence identified CCR2+ cells. Isotype control antibodies and GFP-mice were used to validate the gating strategy. Cell populations were defined using the following markers: CD45⁺CD11b⁺CD64^{int}Ly6C^{high} for monocytes, CD45⁺CD11b⁻CD64⁺Ly6C^{lo} for macrophages, and CD45⁺CD11c⁺CD64-MHCII⁺ for dendritic cells. For CD163, cells were stained with antibodies specific for CD45, CD64, Ly6C, CD163 and MHCII. Isotype control antibodies were used to validate the gating strategy. Cell populations were defined using the following markers: CD45⁺CD64⁺MHCII⁺CD163⁺ for CD163+ macrophages, CD45⁺CD11b⁺CD64^{int}Ly6C^{high} for monocytes, CD45*CD11b⁺CD64*Ly6C^{b} for macrophages, and CD45*CD11c⁺CD64-MHCII⁺ for dendritic cells. FloJo software was used to identify the specific cell type.

### Example 2—Development of CD163 Targeting Agent for Imaging and Therapy

The rupture of atherosclerotic plaques is responsible for the majority of cardiac and cardiovascular events such as ischemic strokes and myocardial infarctions. Vulnerable or rupture-prone plaques are characterized by a large necrotic core, inflammation and signs of impaired tissue repair. Of various immune cells overexpressed within the plaques, macrophages play important role mediating the initiation, progression and complication of atherosclerotic lesions. Plaque macrophages are a phenotypically heterogeneous group, where polarized phenotypes range from classically activated proinflammatory (M1) macrophages to alternatively activated reparatory (M2) macrophages.

In contrast to the proinflammatory macrophages that are considered proatherogenic, the alternatively activated macrophages are antiatherogenic by producing anti-inflammatory cytokines and promoting tissue repair. Thus, modulation of macrophages to a less inflammatory but a more reparative phenotype could be a possible approach to stabilize plaques. This subtype of alternatively activated macrophages called Mhem/M(Hb) macrophages is characterized by the overexpression of CD163, a scavenger receptor for hemoglobin-haptoglobin complexes exclusively expressed on monocytes and macrophages. CD163+ macrophages were considered atheroprotective due to their high expression of the anti-inflammatory cytokine IL-10 and heme degrading enzyme hemeoxygenase, as well as their association with reduced oxidative stress. In Apoe^{-/-} mice, the deficiency of CD163 exhibited increased plaque areas, less complex lesions and thicker fibrous caps. In humans, the expression of CD163 is associated with a vulnerable plaque phenotype. Therefore, the detection of CD163 could be of clinical significance not only to determine the vulnerability of atherosclerotic plaques, but also to provide critical information as a target to manage the treatment. However, there is no molecular agent available for CD163 targeting, which is an unmet need for both preclinical and clinical research.

### Phage Display Screening of CD163 Targeting Ligand

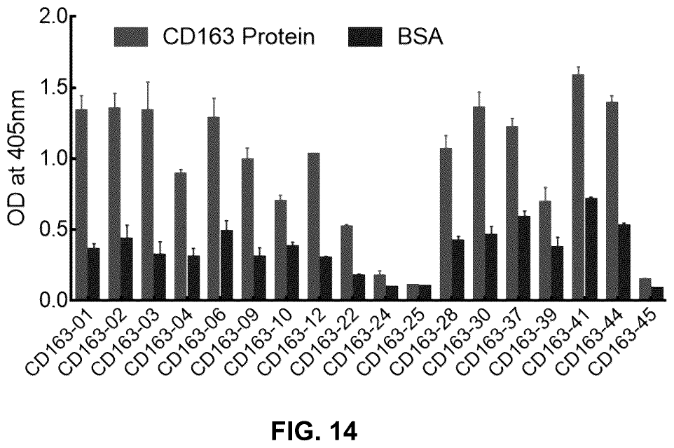

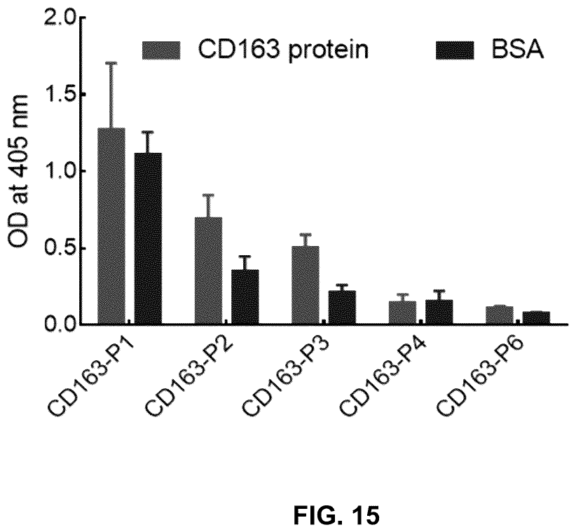

To develop CD163 targeting ligand, phage display screening was performed using Ph.D.-12 Phage Display Peptide Library binding human CD163 protein with bovine serum albumin (BSA) as control. After the duplicated sequences were removed from initial screening, 18 peptide sequences showing specific binding to CD163 with superior selectivity compared to BSA were identified (FIG. 14). The top 5 peptide sequences were then picked and customized them for peptide enzyme-linked immunosorbent assay (ELISA) (FIG. 15), and finally identified one peptide named ICT-01 (WFWNHGWQWSSK) (ICT-01 SEQ ID NO: 1) as candidate for radiotracer development.

### Design of CD163 Targeting Radiotracer

To develop a CD163 binding radiotracer for positron emission tomography (PET) imaging, a 2-[1,4,7-Tiazacyclononan-1-yl-4,7-bis(tBu-ester)]-1,5-pentanedioic acid (NODAGA) based macrocyclic chelator to ICT-01 was conjugated through tryptophan residue on N-terminus via amide bond (NODAGA-ICT-01) (FIG. 9). The CD163 targeted conjugate NODAGA-ICT-01 was then radiolabeled with either ⁶⁴Cu (t_{1/2}=12.7 h, ⁶⁴Cu-ICT-01) or ⁶⁸Ga (t_{1/2}=68 min, ⁶⁸Ga-ICT-01) for in vitro and in vivo applications.

### In Vitro Cell Binding Assay

The in vitro cell binding assay of ⁶⁴Cu-ICT-01 was performed in U87 cells, which overexpresses CD163 receptor. As shown in FIG. 10A, the non-radioactive ICT-01 competes with the radiotracer ⁶⁴Cu-ICT-01 in a concentration dependent manner, leading to a half-maximal inhibitory concentration (IC₅₀) of 26.8 t 21.4 nM (n=3).

### In Vivo Pharmacokinetics

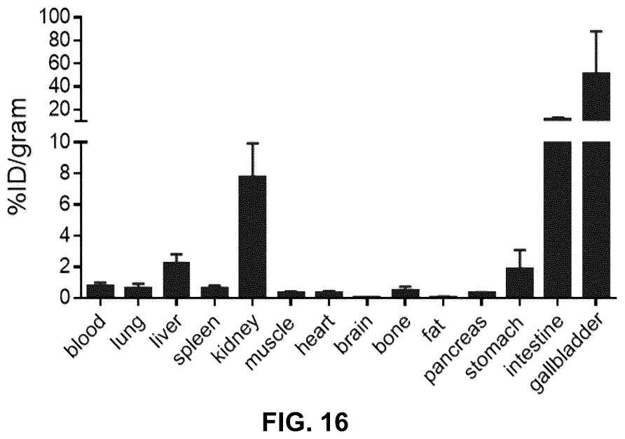

In vivo pharmacokinetics of ⁶⁴Cu-ICT-01 was performed in wild type C57BL/6 mice at 1 h, 2 h, and 4 h post injection via tail vein. As shown in FIG. 10B, ⁶⁴Cu-ICT-01 showed effective renal clearance and low retention in most organs. The blood pool organs (blood, heart, lung) had less than 0.5% ID/gram retention at 1 h post injection while the liver uptake was less than 2% ID/gram at all times. Interestingly, gallbladder showed high retention of ⁶⁴Cu-ICT-01 (31.8±8.1% ID/gram) at 1 h, followed by rapid decrease to less than 4% ID/gram at 4 h. Similarly, the ⁶⁸Ga labeled ICT-01 displayed comparable biodistribution profile at 1 h time point (FIG. 16).

### In Vivo PET/CT Imaging

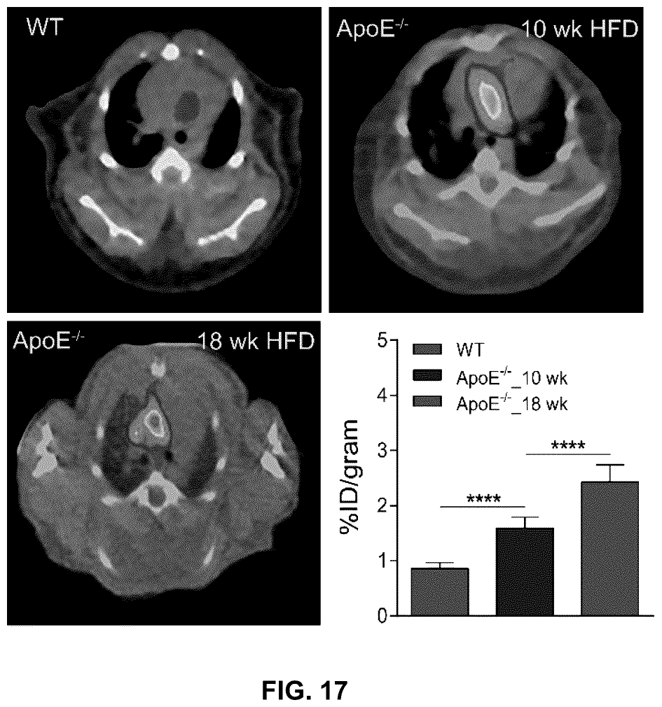

The in vivo imaging performance including sensitivity and specificity of ⁶⁴Cu-ICT-01 was evaluated in multiple animal models. In mouse ApoE^{-/-} atherosclerosis model, ⁶⁴Cu-ICT-01 showed significantly elevated uptake at aortic arch at 10 weeks (1.59±0.19% ID/gram, n=7) post high fat diet (HFD) compared to wild type mice (0.86±0.10% ID/gram, p<0.05, n=7) (FIG. 17), indicating the capability of as-developed radiotracer detecting atherosclerotic plaques. With the progression of atherosclerosis, ⁶⁴Cu-ICT-01 revealed further increased uptake at 18 weeks (2.43±0.27% ID/gram, n=4) post HFD, suggesting the sensitivity of the tracer to track aggravated plaques.

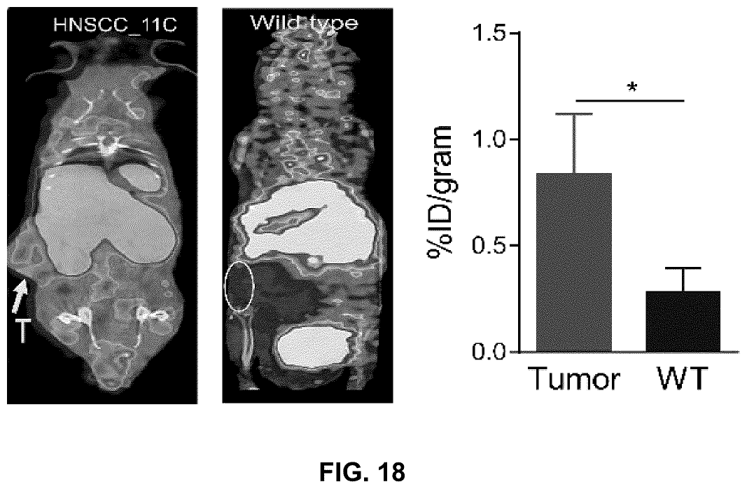

The imaging efficiency of ⁶⁴Cu-ICT-01 detecting CD163 was also assessed in head and neck squamous cell carcinoma (HNSCC) due to its overexpression in tumor-associated macrophaes. As shown in FIG. 18, ⁶⁴Cu-ICT-01 revealed specific detection of PDX_11c tumor while low retention was observed in the wild type NSG mice. Quantitative uptake demonstrated approximately two-fold higher uptake in HNSCC PDX_11c than that determined in a similar location in wild type NSG mice. These data confirmed the tumor imaging efficiency of ⁶⁴Cu-ICT-01

### Example 3 Development of CD163 Targeted PET Radiotracer Imaging Resident Macrophages in Atherosclerosis (Manuscript)

This example demonstrates the efficacy of a CD163 radiotracer disclosed herein.

### Abstract

Purpose: Tissue resident macrophages are complementary to proinflammatory macrophages to promote the progression of atherosclerosis. The non-invasive detection of their presence and dynamic variation will be important to understand their role in the pathogenesis of atherosclerosis. The goal of this study was to develop a targeted PET radiotracer for imaging CD163+ macrophages in multiple mouse atherosclerosis models and assess the potential of CD163 as a biomarker for atherosclerosis in humans.

Methods: CD163 binding peptide was identified using phage display and conjugated with NODAGA chelator for ⁶⁴Cu radiolabeling (⁶⁴Cu-ICT-01). CD163 overexpressing U87 cells were used to measure the binding affinity of ⁶⁴Cu-ICT-01. Biodistribution studies were performed in wild type C57BL16 mice at multiple time points post tail vein injection. The sensitivity and specificity of ⁶⁴Cu-ICT-01 imaging CD163+ macrophages upregulated on the surface of atherosclerotic plaques were assessed in multiple mouse atherosclerosis models. Immunostaining, flow cytometry, and single cell RNA sequencing (scRNAseq) were performed to characterize the expression of CD163 on tissue resident macrophages. Human carotid atherosclerotic plaques were used to measure the expression of CD163+ resident macrophages and test the binding specificity of ⁶⁴Cu-ICT-01.

Results: ⁶⁴Cu-ICT-01 showed high binding affinity to U87 cells. Biodistribution study showed rapid blood and renal clearance with low retention in all major organs at 1 h, 2 h, and 4 h post injection. In an ApoE^{-/-} mouse model, ⁶⁴Cu-ICT-01 demonstrated sensitive and specific detection of CD163+ macrophages and capability to track the progression of atherosclerotic lesions, which was further confirmed in Ldlr−/− and proprotein convertase subtilisin/kexin type 9 (PCSK9) mouse models. Immunostaining showed elevated expression of CD163+ macrophages across the plaques. Flow cytometry and scRNAseq confirmed the specific expression of CD163 on tissue resident macrophages. Human tissue characterization demonstrated high expression of CD163+ macrophages on atherosclerotic lesions and ex vivo autoradiography revealed specific binding of ⁶⁴Cu-ICT-01 to human CD163.

Conclusion: This work reported the development of a PET radiotracer binding CD163+ macrophages. The elevated expression of CD163+ resident macrophages on human plaques indicated the potential of CD163 as a biomarker for vulnerable plaques. The sensitivity and specificity of ⁶⁴Cu-ICT-01 imaging CD163+ macrophages warrant further investigation in translational settings.

### Materials & Methods

Materials were purchased from Sigma-Aldrich and used without further purification unless otherwise stated. The ⁶⁴Cu (half-life=12.7 h, β⁺=17%, β⁺=40%) was produced at the Washington University cyclotron facility. NODAGA-tris(t-Bu ester) chelator was purchased from Macrocyclics. The CD163 targeting peptide ICT-01 was customized by Ke Biochem. Reversed-phase high-performance liquid chromatography (HPLC) was performed on an Agilent 1200 system equipped with a photo diode array detector and a radioisotope detector (B-FC-3200; BioScan Inc.) on a C-18 analytical column (5 μm, 4.6×250 mm; Thermo Fisher). Water (18 MD-cm) was obtained from a Milli-Q water filtration system (Millipore Corp., Bedford, MA)

### Mouse Atherosclerosis Models

All animal studies were performed in compliance with guidelines set forth by the National Institutes of Health Office of Laboratory Animal Welfare and approved by the Institutional Animal Care and Use Committee at Washington University. The mouse atherosclerosis models were induced in ApoE^{-/-} or Ldlr^{-/-} mice (Jackson Laboratory) around 8 weeks old using high fat diet (HFD, Teklad). For the PCSK9 murine atherosclerosis model, wild type (WT) C57BL/6 mice were injected with 5.0×10¹¹ genome copies of adeno-associated virus 2 vector encoding murine PCSK9 (proprotein convertase subtilisinlkexin type 9; AAV-mPCSK9 vector, Vector Biolabs) via tail vein at 5 to 6 weeks old and then fed HFD. Both male and female mice were used in these studies.

### Phage Display Screening

Phage display screening of CD163 targeting peptides was performed with a Ph.D.-12™ library (100 μL, 1×10¹³ plaque forming units (pfu)/mL) purchased from New England Biolabs (Ipswich, MA) and human CD163 protein with His tag (Acrobiosystems INC, Newark, DE). Following three rounds of biopanning, approximately 50 plaques were collected for Sanger DNA sequencing and enzyme-linked immunosorbent assay (ELISA). The Sanger DNA sequencing was performed by GENEWIZ from Azenta Life Sciences by using -96111 primer (5′-CCCTCATAGTTAGCGTAACG-3′) from IDT (Coralville, Iowa) and analyzed with BioEdit 7.2. Based on the sequencing data, eighteen peptide sequences were identified for phage ELISA to further screen peptides with optimal binding to CD163. Of these, five peptide sequences were identified and customized with biotin label by Ke Biochem. These biotinylated peptides were screened against CD163 protein using ELISA to select candidate peptide sequences for radiotracer development.

### Synthesis and Radiobaeling of ICT-01

The synthesis of CD163 targeted ICT-01 was performed by conjugating NODAGA chelator to the N-terminus of ICT-01 peptide via an amide bond. Briefly, DIPEA (6 equivalence) was added to DMF solution of NODAGA-tris(tBu ester) (2 equivalence) and HATU (2 equivalence). After incubation for 15 min (500 rpm, RT), the resulting solution was mixed with ICT-01 peptide (with the side-chain protecting groups, 1 equivalence). Following incubation overnight (500 rpm, RT), water was added to the solution and the resulting precipitate was collected by centrifugation. After washing with water, lyophilization afforded the crude conjugate. Protecting groups were removed by incubation with the cocktail TFA:TIPS:DTT:water (88:2:5:5). Progress of deprotection was monitored by HPLC. The resulting mixture was added to cold diethyl ether to induce precipitation of NODAGA-conjugated ICT-01. Dried precipitate after centrifugation was dissolved in acetonitrile/water and lyophilization yielded the crude NODAGA-conjugated ICT-01. If necessary, the resulting lyophilized powder was re-dissolved in 0.1% acetic acid and re-lyophilized to further complete the deprotection reaction. The crude conjugate was purified by HPLC with water and acetonitrile containing 0.1% TFA as eluents and characterized by ESI-MS (m/z: [M+2H]²⁺ Calcd for C₉₇H₁₂₃N₂₅O₂₃ 1002.9606; Found 1002.9579). The radiolabeling of ICT-01 with ⁶⁴Cu was carried out in sodium acetate buffer (20 mM, pH 7) following an established procedure.

### In Vitro Cell Binding Assay

An in vitro cell binding assay of ⁶⁴Cu-ICT-01 to CD163 was performed with a CD163 overexpressing U87 cell line. U87 cells seeded on a 12-well tissue culture plate were incubated at room temperature with 37 kBq of ⁶⁴Cu-ICT-01 in the presence of non-radioactive ICT-01 in increasing concentrations (10⁻⁵-10⁻¹⁰ M) in binding media (Dulbecco's Modified Eagle Medium (DMEM): 25 mM HEPES, 0.2% bovine serum albumin (BSA), and 0.3 mM 1,10-phenanthroline). The reaction media was aspirated after incubation. Cells were rinsed with 0.8 mL of ice-cold pH 7.4, 0.2% BSA/0.01 M phosphate buffered saline (PBS) twice and lysed in 0.8 mL of 1 M NaOH for 10 min. Each well was rinsed with 0.8 mL of PBS (1×, pH7.4)/0.2% BSA, which was collected into a plastic tube. The radioactivity in tubes was measured with a gamma counter (Wallac Wizard 1480, Perkin Elmer). The half-maximal inhibitory concentration (IC₅₀) for ⁶⁴Cu-ICT-01 was calculated using the GraphPad Prism (10.0.2).

### Biodistribution Studies

Radiolabeled ⁶⁴Cu-ICT-01 with 95% or greater radiochemical purity was reconstituted in 0.9% sodium chloride (APP Pharmaceuticals). Wild type (WT) C57BL/6 mice (male, 8-10 weeks old, 20-25 g, n=4/group) anesthetized with inhaled isoflurane were injected with approximately 370 kBq of ⁶⁴Cu-ICT-01 in 100 μL saline via tail vein. The mice were reanesthetized before euthanization by cervical dislocation at 1 h post injection. Organs of interest were collected, weighed, and counted in a well gamma counter (Beckman 8000). Standards were prepared and measured along with the organ tissue to calculate the percentage injected dose per gram of tissue (% ID/gram).

### PET/CT

Dynamic PET scans were obtained using an Inveon MM PET/CT (Siemens, Malvern, PA) or Nanoscan PET122S/CT1512 in vivo imager (Mediso, Arlington, VA) following tail vein injection of ⁴Cu-ICT-01 (3.7-7.4 MBq per mouse). The PET images were collected at 45 to 60 minutes post injection to minimize the effect of radiotracer blood retention on atherosclerotic plaque uptake. CT scan (low magnification, 4×4 binning, 4096 px transaxial CCD size, 133 mm Axial scanning length, 60 kV, 500 μA, 300 ms exposure, sum 1 frame) was performed simultaneously for anatomic registration. Competitive receptor blocking studies (ICT-01: ⁶⁴Cu-ICT-01 molar ratio=100:1) were performed in ApoE^{-/-} mice at 30 weeks on HFD. PET images were corrected for attenuation, scatter, normalization, and camera dead time, and co-registered with CT images. The two scanners were cross-calibrated periodically. PET images were reconstructed with the maximum a posteriori algorithm. Radiotracer uptake at the aortic arch was calculated as standardized uptake value (SUV) in three-dimensional regions of interest from PET images without correction for partial volume effect using Inveon Research Workplace software (Siemens).

### Histology and Immunofluorescence Staining

Following PET/CT, the hearts and aortic arches of atherosclerotic mice were perfusion-fixed in situ with freshly prepared 4% paraformaldehyde in 1×PBS for hematoxylin and eosin (H&E) and immunofluorescent staining. Serial sections of 5 μm thickness were cut from paraformaldehyde-fixed (24 h), OCT-embedded specimens. Blocking serum was added (10% donkey serum in PBS-T) for 1 h to prevent non-specific binding. The sections were then incubated overnight at 4° C. with primary antibodies (anti-CD163, 1:100 in 1% blocking serum; ThermoFisher). Sections were washed in PBS, and secondary antibodies were applied for 1 h (donkey anti-rat Cy5, 1:300, Jackson ImmunoResearch Laboratories, West Grove, PA). Sections were washed in PBS and coverslipped with DAPI mounting medium. Control staining was performed without primary antibodies. All sections were imaged using a Leica THUNDER Imager 3D tissue microscope system. CD163+ macrophages were manually counted from multiple 500 μm² area on the plaques.

### Flow Cytometry

Saline perfused aortas were minced and digested in DMEM containing Collagenase IV (450 U/mL), DNAse I (60 U/mL), and Hyaluronidase I-s (60 U/mL) for 45 minutes at 37° C. to create single cell suspensions. To deactivate the digestion, samples were washed with Hank's balanced salt solution (HBSS) supplemented with 2% fetal bovine serum (FBS) and 0.2% BSA. Cells were filtered through 70 μm cell strainers and resuspended in ammonium-chloride-potassium (ACK) lysing buffer (Thermo Fisher Scientific) for 2 minutes to perform red blood cell lysis prior to washing with DMEM and resuspending in 1 mL fluorescence-activated cell sorting (FACS) buffer (Invitrogen eBioscience Flow Cytometry Staining Buffer). Cells were stained with monoclonal antibodies at 4° C. for 15 minutes in the dark. All antibodies were obtained from Biolegend: CD45-PerCP/Cy5.5 clone 30-F11, CD11b-BV785 clone M1/70, Ly6C-BV510 clone HK1.4, Ly6G-BV605 clone 1A8, CD64-APC clone X4-5/7.1, CD163-PE clone S150491, and MHCII-APC/Cy7 clone M5/114.15.2. Cells were washed and resuspended in 300 μL of FACS buffer to remove unbound antibodies from analysis. Flow cytometric analysis was performed on the Cytek Aurora platform and subsequent data analysis was performed on FlowJo. Macrophages were gated as CD45+CD11b+CD64+Ly6C^{lo} cells and CD163+ macrophages were further gated as CD163+MHCII^{hi} cells.

### Single Cell RNA Sequencing

Single cell suspensions were generated from the aorta as described in the flow cytometry section. Cells were stained with 1 μL CD45-PerCP-Cy 5.5 and incubated for 30 minutes before sorting CD45+/DAPI⁻ cells. DAPI (BD Biosciences, 564907) was added before sorting. Collected cells were then recentrifuged and resuspended in cell resuspension buffer to a target concentration of 1000 cells/μL. Cells were counted on a hemocytometer and concentration adjusted as necessary.

The Chromium Single Cell VDJ 3′ Reagent V3.1 Kit from 10× Genomics was used to capture the transcriptome of individual cells. For each of the samples, 10,000 cells were loaded into a single well of a Chip G kit for GEM generation. Reverse transcription, barcoding, cDNA amplification, and purification were performed according to the Chromium 10× 3′ v3.1 protocol as per the 10× genomics protocol. Libraries were made using the 10× 3′v3.1 chemistry at McDonnel Genome Institute within Washington University. Each sample was sequenced on a NovaSeq 6000 S4 platform with a sequencing depth of approximately 20,000-30,000 reads per cell. Sequencing reads were aligned to the whole genome pre-mRNA reference generated from the mm10 transcriptome using Cell Ranger v6.1 software (10× Genomics) according to the 10× Genomics instructions. The count matrices were pre-processed and analyzed using R package Seurat (v4). Cells with fewer than 1,000 or greater than 25,000 UMI counts, as well as cells with greater than 10% mitochondrial read percentage, were excluded from all subsequent analysis. Scrublet was used to quantify doublet scores and cells with a score above 0.25 were excluded from downstream analysis.

For each sample, counts were transformed and normalized using SCTransform with default thresholds. Principal component analysis (PCA) was performed on the integrated object using RunPCA and 50 components were used for subsequent analysis. Non-linear dimensional reduction and visualization were performed using RunUMAP. Cells showing co-expression of multiple cell-type-specific genes were identified as doublets and removed from any downstream analysis. Differentially expressed genes were identified using the FindAllMarkers function and genes with a log 2 fold change >0.58 and adjusted p-value <0.05 were deemed statistically significant. To perform cell type specific analysis, cell types were clustered into cell states using the approach described above. DE analysis was also performed between the saline and PD1 treated groups. Statistically significant genes were used for pathway analysis using EnrichR. For resident macrophages a z-score was computer using Seurat and Nebulosa was used for gene expression density. Published human coronary artery scRNA-seq (GSE131780) were downloaded and processed as above—a resident macrophage gene z-score was calculated and plotted in the global UMAP in Seurat.

### Autoradiography of Human Carotid Arteries

De-identified, paraffin-embedded human carotid artery specimens were acquired from the Vascular Surgery Department Bio Bank under Institutional Review Board protocol (#201911199) approved by the Human Research Protection Office of Washington University. The ex vivo tissue binding of ⁶⁴Cu-ICT-01 to human CD163 was performed following established procedures. All autoradiography were collected with a GE Typhoon FLA 9500 Variable Mode Laser Scanner at 50-micron resolution. ImageJ software was used for the quantification of ⁶⁴Cu-ICT-01 uptake in carotid artery samples. Multiple regions of interest were drawn within the entire tissue section for correlation analysis with CD163 immunostaining. Moreover, a MATLAB (MathWorks, Natick, MA) based program was implemented to create a whole tissue correlation heatmap between CD163 immunostaining images and autoradiography images. Upon importing a grayscale CD163 immunofluorescence stained image (IF) and its corresponding autoradiography image (AR) as matrices, the software applied a Gaussian filter with a sigma value of 2 to the IF. Both images were thresholded and binarized, before a multimodal geometric transformation object warped the AR image to match the fixed IF image. Upon image registration, a new image matrix for the correlation heat map was generated with the same dimensions as the respective IF and AR images. Both IF and AR images were divided into 40,000 blocks, and non-zero pixel values within each block were filtered using an impulse response filter prior to correlation. The resulting correlation coefficient was used to fill all matrix values in the new image matrix corresponding to the dimensions of the current correlation block. The software iterated through all correlation blocks to generate the correlation heat map matrix.

### Statistical Analysis

Group variation is described as mean±SD. Groups were compared using 1-way ANOVA with a Tukey adjustment using GraphPad (Prism, 10.0.2). Individual group differences were determined using a 2-tailed Mann-Whitney test. The significance level in all tests was a P value less than 0.05.

### Results

### Synthesis, In Vitro Cell Binding Assay, and Biodistribution

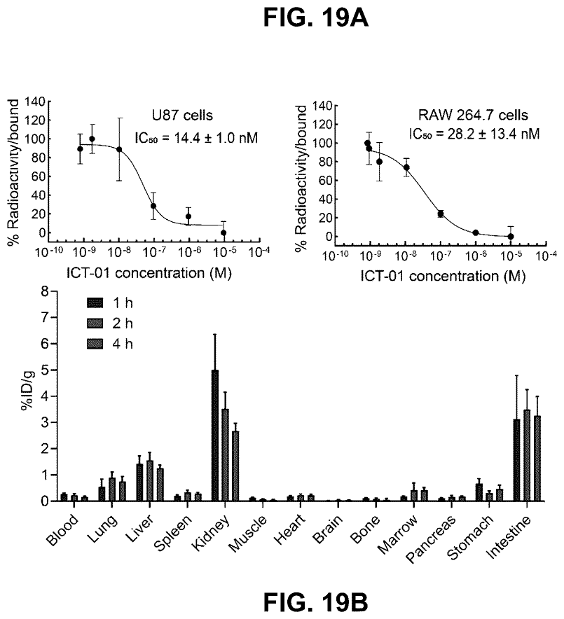

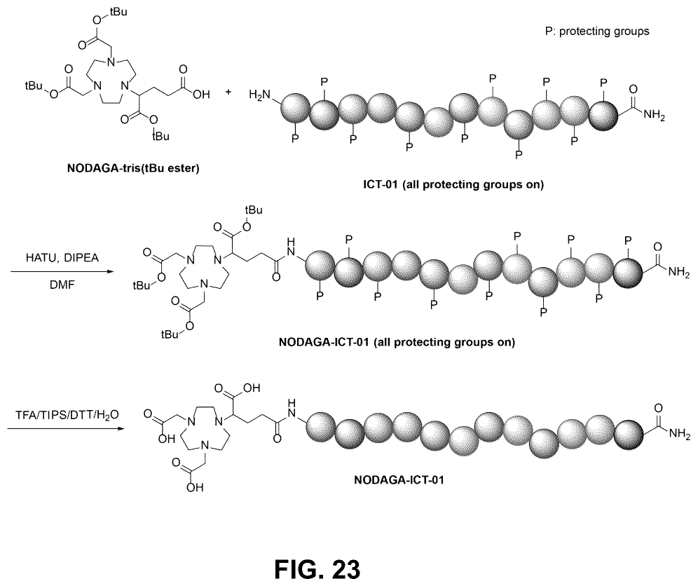

The ICT-01 conjugate was characterized with HPLC-MS, which showed chemical purity and the conjugation of one NODAGA chelator per ICT-01 peptide (FIG. 23). Radiolabeling with ⁶⁴Cu showed high specific activity (⁶⁴Cu-ICT-01, 111 MBq-185 MBq/nmol), enabling tracer amount administration for in vivo imaging. ⁶⁴Cu-ICT-01 in vitro binding assay in U87 cells showed specific and high binding affinity with IC₅₀ determined as 14.4±1.0 nM (FIG. 19A, n=3).

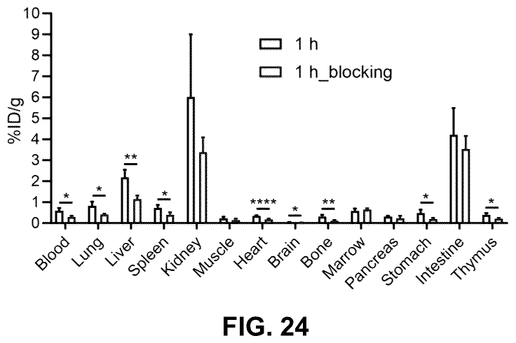

In vivo pharmacokinetic evaluation of ⁶⁴Cu-ICT-01 was performed in WT C57BL/6 mice at 1 h, 2 h, and 4 h post injection via tail vein. As shown in FIG. 19B, ⁶⁴Cu-ICT-01 demonstrated effective renal and blood clearance, leading to low retention in most organs. Both blood and heart had less than 0.5% ID/gram retention at all the time points. The hepatic uptake was always less than 2% ID/gram accumulation during the 4 h collection while a similar pattern was also observed for intestine. In spleen, the tracer uptake was constantly lower than 0.5% ID/gram all the time points. Additionally, ⁶⁴Cu-ICT-01 biodistribution blocking studies were performed at 1 h post injection (FIG. 24).

The significantly decreased tracer uptake in liver, spleen and thymus indicated its targeting specificity.

PET Imaging of CD163 in ApoE^{-/-} and LDLR^{-/-} Mice Atherosclerosis Models

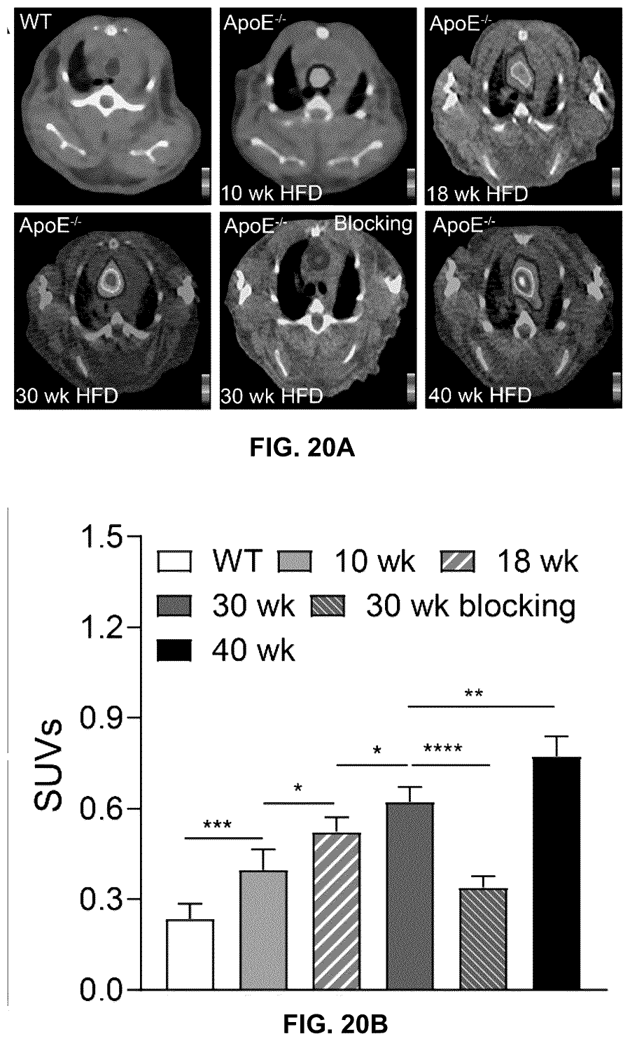

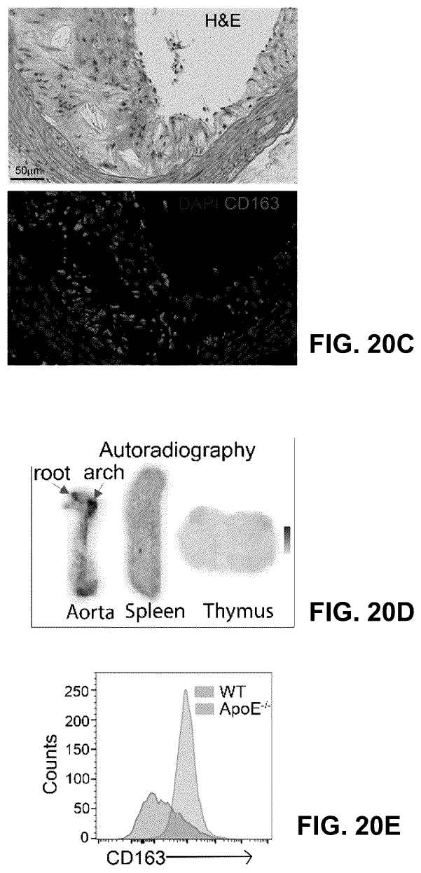

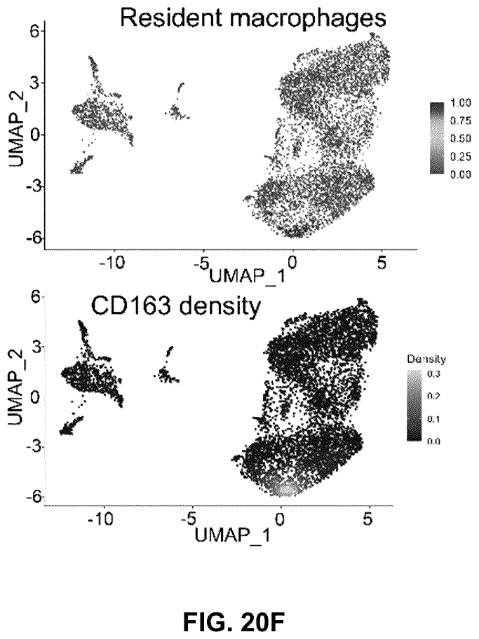

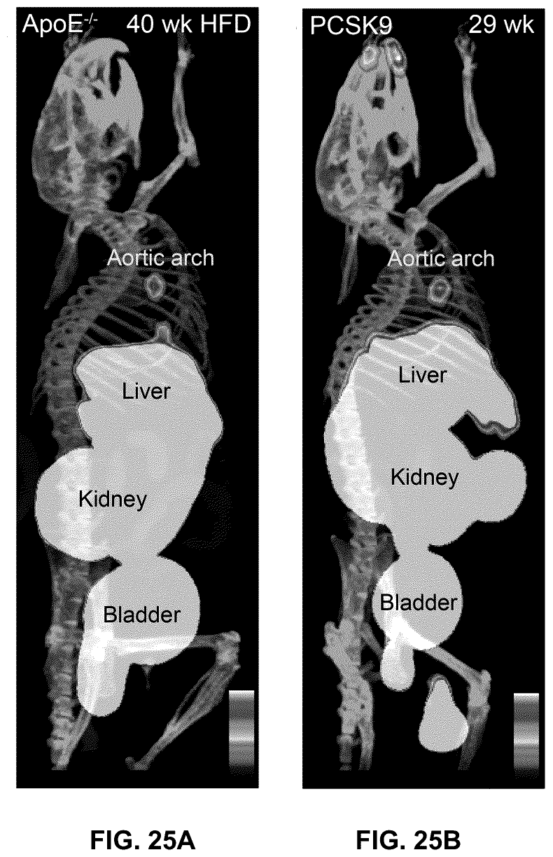

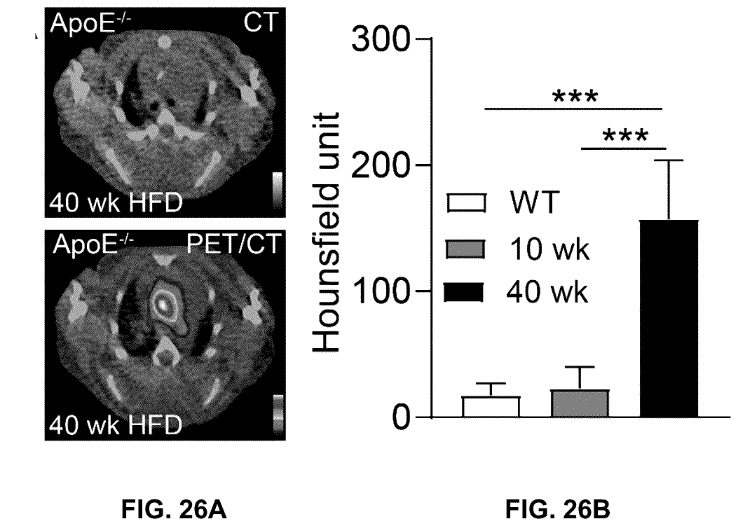

To assess the potential of ⁶⁴Cu-ICT-01 imaging atherosclerotic plaques, a time course study in ApoE^{-/-} mice from 10 weeks to 40 weeks on HFD was performed. In contrast to the minimal tracer retention within the aortic arch of WT mice (SUV=0.24±0.05, n=7), ⁶⁴Cu-ICT-01 revealed a significantly increased tracer uptake at aortic arch (SUV=0.40±0.07, p=0.0001, n=7) after 10 weeks' HFD (FIGS. 20A-B). With the progression of atherosclerotic lesions, the tracer uptake at aortic arch increased overtime, leading to more than 2 times higher uptake (SUV=0.77, p<0.0001, n=4) at 40 week's post HFD (FIGS. 20A-B), which was clearly illustrated in the maximum intensity projection (MIP) image (FIG. 25A). Moreover, the intense PET signals were next to the microcalcification within aortic arch visualized by CT (FIG. 26). The coincidence between PET and CT for ApoE^{-/-} mice at 40 weeks post HFD suggested the specific binding of ⁶⁴Cu-ICT-01 to plaques. Importantly, the competitive receptor blocking study performed at 30-week post HFD reduced the PET signals by approximately 50% (p<0.0001, n=4-6), confirming the targeting specificity of ⁶⁴Cu-ICT-01.

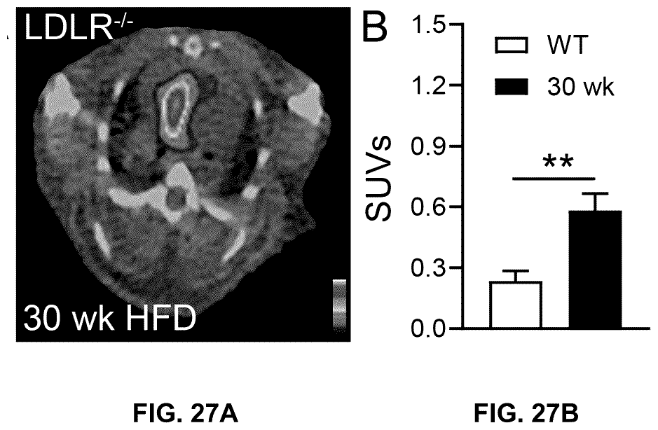

In Ldlr^{-/-} mice at 30-week post HFD, ⁶⁴Cu-ICT-01 revealed strong tracer uptake (SUV=0.58±0.09, n=6) at aortic arch (FIG. 27), which was comparable to that acquired in ApoE^{-/-} mice (SUV=0.62±0.05, n=6) at same time point, further validating the plaque targeting efficiency of ⁶⁴Cu-ICT-01.

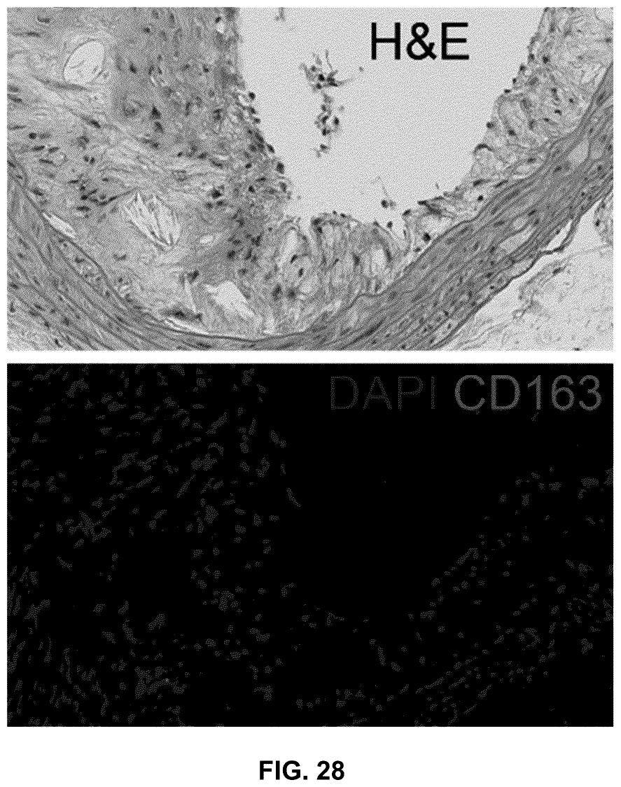

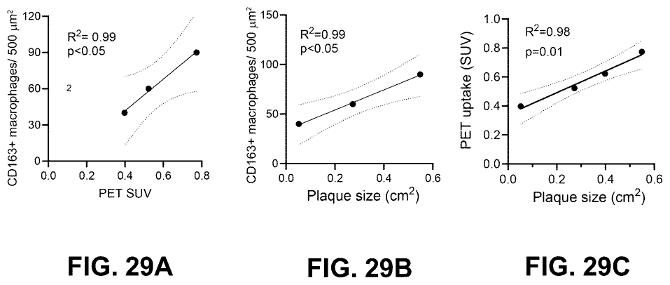

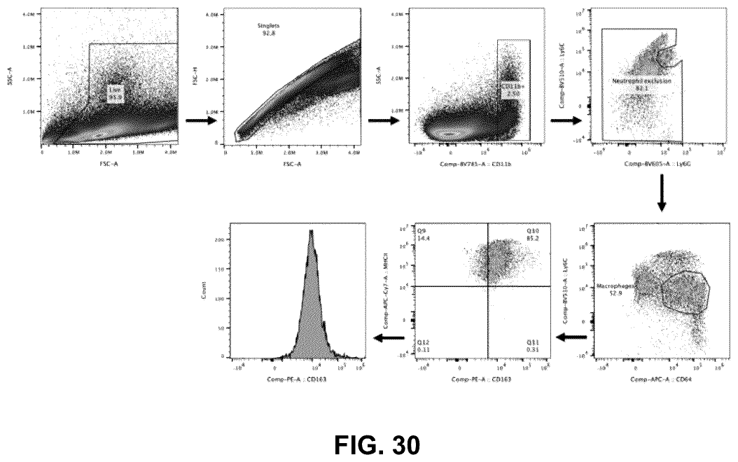

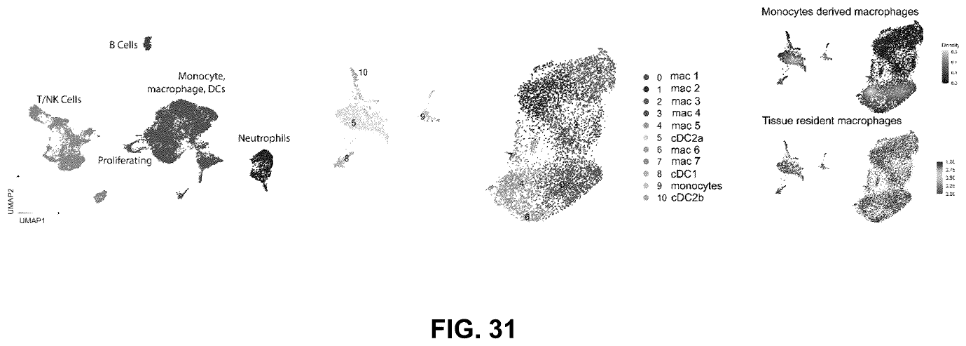

H&E staining of aortic arch collected from ApoE^{-/-} mouse after 20 weeks HFD showed significant development of plaques including enlarged neointima, infiltration of inflammatory cells, and large lipid pool. Immunofluorescent staining revealed the extensive expression of CD163+ macrophages throughout the plaques (FIG. 20C), which was further validated by the little signals observed from the control immunofluorescent staining (FIG. 28). Quantification of immunofluorescent staining demonstrated gradually increased expression of CD163+ macrophages along the progression of atherosclerotic lesions, which showed linear correlations with ⁶⁴Cu-ICT-01 uptake at aortic arch and plaque size, respectively, during the time course study. Owing to the high expression of CD163+ macrophages on the plaques, ⁶⁴Cu-ICT-01 uptake also correlated well with plaque size during the 40-week study (FIG. 29). Ex vivo autoradiography performed in ApoE^{-/-} mice at 20 weeks post HFD revealed specific tracer uptake at aortic arch and root with low retentions observed in both spleen and thymus (FIG. 20D). Consistent with immunostaining data, flow cytometry demonstrated significantly higher expression of CD163+ macrophages on the aortas collected from ApoE^{-/-} mice at 20 weeks post HFD than those collected from WT mice (FIG. 20D and FIG. 30). Moreover, single cell RNA sequencing of aortic arteries from ApoE^{-/-} mice after 20 weeks' HFD confirmed that CD163 is highly expressed with high density in a subtype of resident macrophage classified by resident macrophage specific genes including Folr2, Lyve1, and F13a1 (FIG. 20E and FIG. 31). These data highlighted the specific expression of CD163 on tissue resident macrophages in atherosclerotic lesions and the potential of CD163 as a biomarker for plaque development. The sensitive detection of CD163+ macrophages in ApoE^{-/-} mice suggested the capability of ⁴Cu-ICT-01 for noninvasively tracking the progression of atherosclerotic lesions.

### PET Imaging of CD163 in PCSK9 Mouse Atherosclerosis Model

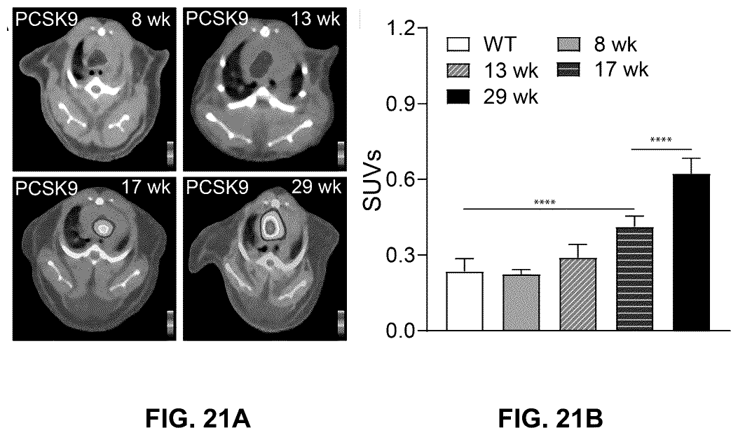

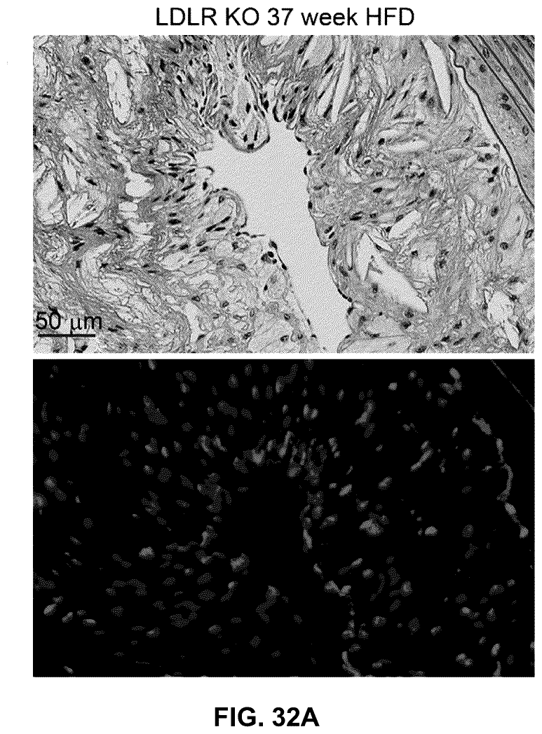

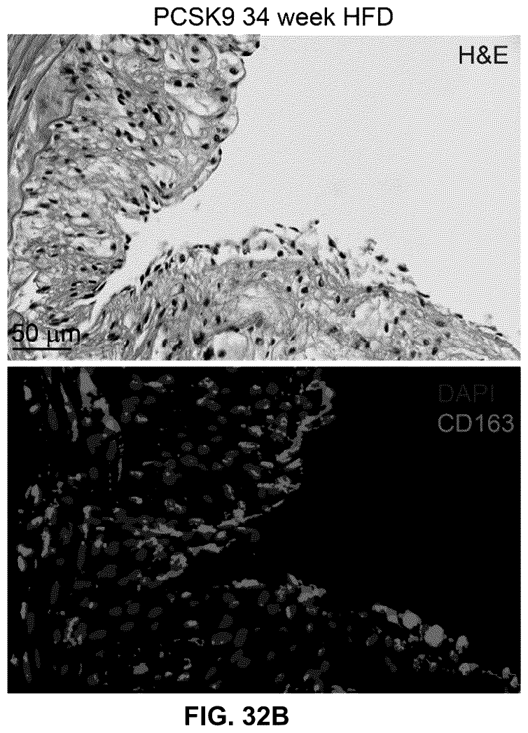

In addition to the genetically engineered mouse atherosclerosis models including ApoE^{-/-} and LDLR⁴ mice, the plaque targeting efficiency of ⁶⁴Cu-ICT-01 in a mouse atherosclerosis model was also assessed using WT C57BL16 mice treated with adenoassociated virus 2 vector encoding murine PCSK9. In contrast to the WT mice, PCSK9 mice at 8 week (SUV=0.22±0.02, n=4) and 13 week (SUV=0.29±0.05, n=8) post HFD did not show any increased tracer accumulation (FIG. 21), consistent with the time course of atherosclerotic lesion development in this model. At 17 weeks post HFD, ⁶⁴Cu-ICT-01 showed substantially intensified tracer localization (SUV=0.41±0.04, p<0.0001, n=8) at aortic arch, which further increased to approximately 3 times (SUV=0.62±0.06, p<0.0001, n=4) as much as that in WT mice after 29 weeks' HFD (FIG. 21). The strong tracer uptake within aortic arch was also depicted in the MIP image (FIG. 25B). Moreover, consistent with the histopathological features of the plaques in ApoE^{-/-} mice, significant development of atherosclerotic lesions and expression of CD163+ macrophages were also determined in the aortic arch of PCSK9 mouse at 34-week post HFD (FIG. 32), supporting the PET imaging results.

### Assessment of CD163 Expression on Human Atherosclerotic Specimens

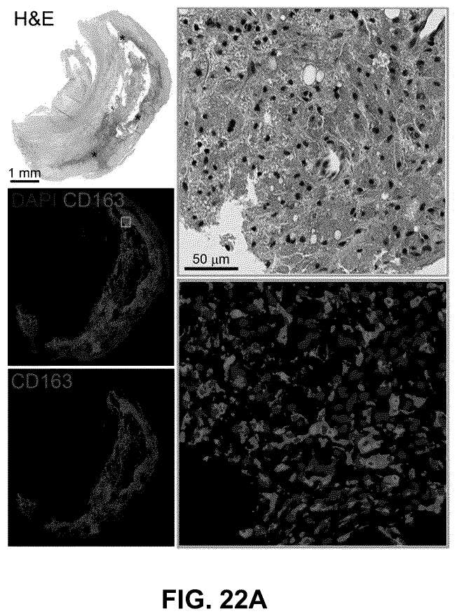

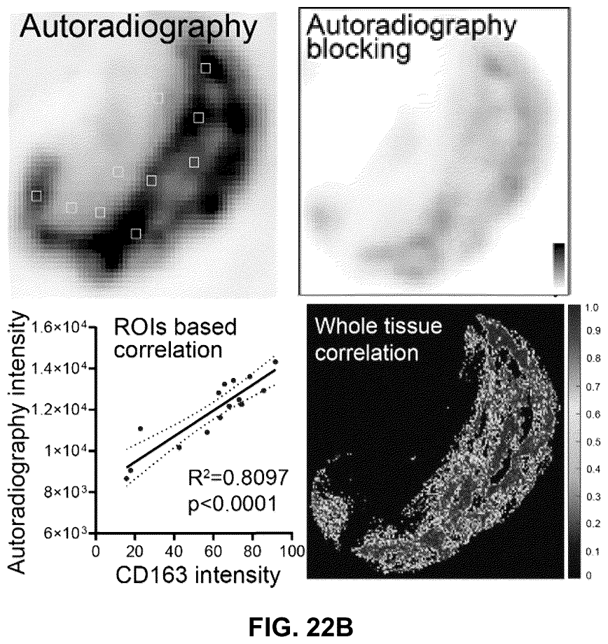

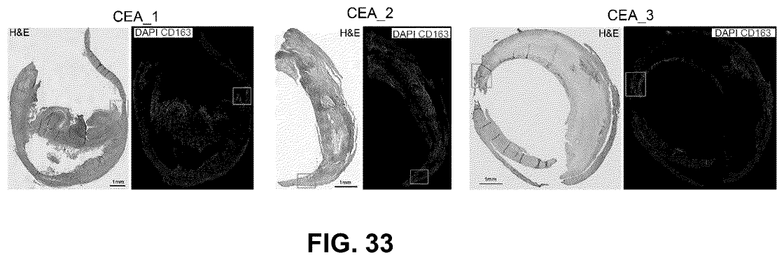

To assess the translational potential of CD163 as a biomarker for human atherosclerosis, human plaque specimens collected from carotid endarterectomy (CEA) were characterized to assess its expression levels and related histopathological features. H&E staining of a maximally diseased portion of a CEA specimen showed large fibrous capsule, necrotic core, significant calcification, substantial intraplaque hemorrhage, and high infiltration of inflammatory cells. Immunofluorescent staining showed high abundance expression of CD163+ macrophages across the tissue with most signals associated with the infiltrated inflammatory cells and intraplaque hemorrhage but not the fibrous capsule (FIG. 22A). Characterization of additional CEA tissues demonstrated the high expression of CD163+ macrophages on both maximally and minimally diseased portions. Particularly, intense accumulation of CD163+ macrophages was observed at the shoulder regions of these CEA specimens independent of the difference of histopathological features (FIG. 33). The potential of ⁶⁴Cu-ICT-01 binding to human CD163 was assessed via ex vivo autoradiography following established protocol. As shown in FIG. 22B, ⁶⁴Cu-ICT-01 revealed intensive and heterogeneous binding to the CEA specimen. Moreover, both ROIs based and whole tissue analyses of the coregistered images demonstrated linear correlation between ⁶⁴Cu-ICT-01 autoradiography and CD163 immunostaining of the CEA specimen, suggesting the binding specificity of ⁶⁴Cu-ICT-01 to human CD163.

### Discussion

The complex pathophysiology of atherosclerosis reflects a systemic proinflammatory process involving innate and adaptive immune responses engaging multiple immune cells such as monocytes, macrophages, and T cells. The multiplex interplay between these processes is characterized by chronicity but with dynamic variation that yields plaque lesions with constantly evolving biologic and anatomic features that determine symptomatology and the induction of clinical events. Most importantly, the complicated pathogenesis of atherosclerosis precludes clinically available anatomic imaging modalities from reliably visualizing specific cellular features or biological processes that identify high-risk individuals for immunomodulatory or targeted treatment.

Of various immune cells, macrophages are pivotal in the chronic inflammatory processes that drive the pathogenesis of atherosclerosis, which makes them the mostly studied immune cells for atherosclerosis diagnosis. It is known that macrophages are hallmarked by phenotypic heterogeneity and express a spectrum of activational programs that exist as a function of their immediate surroundings, which highlighted the importance of imaging the specific subtype of macrophages. Previously, it has been reported that the targeted PET imaging of monocyte-derived proinflammatory CCR2+ macrophages that are instrumental to the plaques' initiation, progression, and complication. However, the anti-inflammatory tissue resident macrophages that maintain macrophages polarization balance and are complementary to the proinflammatory macrophages in promoting plaques development are insufficiently studied. Although some radiotracers were reported targeting resident macrophages, their imaging efficiency and targeting specificities to this subtype of macrophages, especially in humans needs further validation.

Due to the specific expression of CD163 on a subtype of tissue resident macrophages and their exclusive function to clear hemoglobin-haptoglobin complexes following hemorrhage, the presence of CD163+ macrophages is closely associated with vulnerable phenotype of human carotid plaques, making it an unique target to assess plaquw vulnerability. Therefore, a CD163 targeted PET radiotracer based on a 12-mer peptide screened from a Ph.D.™-12 phage display peptide library was developed. In contrast to other PET radiotracers used for CVD imaging, peptide-based radiotracers have the unique advantages including rapid blood and renal clearance, low non-specific retentions in major organs, which may facilitate the realization of improved target-to-background contrast ratio. As demonstrated in the in vitro binding assay and biodistribution studies, the high binding affinity and low blood retention of ⁶⁴Cu-ICT-01 may enable the detection of small atherosclerotic lesions during the initiation of plaques and sensitive tracking of plaque progression. Among various positron emitters for NODAGA radiolabeling, the relative short positron range of ⁶⁴Cu makes it favorable for cardiovascular applications. Additionally, its 12.7 h decay half-life enables the shipping of ⁶⁴Cu radiolabeled tracers to a second site for preclinical or clinical studies.

Using three mouse models of atherosclerosis, it has been demonstrated that ⁶⁴Cu-ICT-01 specifically detects the accumulation of CD163+ macrophages overexpressed on the surface of plaque within aortic arch. The significantly decreased tracer uptake via competitive receptor blocking confirmed its plaque targeting specificity. Moreover, the high binding affinity of ⁶⁴Cu-ICT-01 and favorable pharmacokinetics enabled the sensitive detection of the subtle change of CD163+ macrophages population and plaque development in both ApoE^{-/-} and PCSK9 mice, which was demonstrated by the linear correlation between ⁶⁴Cu-ICT-01 uptake at aortic arch and CD163 expression, as well as the plaque size during the longitudinal studies.

Immunostaining of atherosclerotic mouse aortas revealed extensive signals of CD163+ macrophages from early lesions to late-stage complex plaque structures. The increased expression of CD163+ macrophages along the progression of atherosclerosis and correlation with plaque sizes indicated the importance of CD163+ macrophages in plaques development. Moreover, flow cytometry and scRNAseq data confirmed the specific expression of CD163 on the tissue resident macrophages, highlighting the significance of this macrophage subset in atherosclerosis pathogenesis. Importantly, the high sensitivity and specificity of ⁶⁴Cu-ICT-01 detecting CD163+ macrophages to track the progression of atherosclerotic lesions, underscoring the potential of this radiotracer for further investigation.

Comparing to preclinical research, human tissues validation of a specific biomarker expression is critical to assess its translational potential. Using the de-identified human CEA specimens, an elevated expression of CD163 on atherosclerotic lesions has been demonstrated. The high accumulation of CD163+ macrophages at the shoulder regions of the plaques and association with intraplaque hemorrhage was consistent with previous reports, which suggested the correlation of CD163 expression with plaque vulnerability. Moreover, ex vivo characterization of human CEA specimens demonstrated the higher expression of CD163+ macrophages in symptomatic patients than that in asymptomatic patients, which highlighted the potential of CD163 as a biomarker to assess plaque vulnerability.

All the mouse atherosclerotic models used require a HFD that does not reflect human-like lipid profile. Although severe atherosclerotic lesions were observed in these models, none of the plaques ruptured. Future studies will need to be performed in rupture-prone animal models to assess the potential of ⁶⁴Cu-ICT-01 detecting plaque vulnerability. Another limitation is that only limited numbers of human CEA specimens were used to assess the expression of CD163+ macrophages. A large cohort of retrospective CEA tissues integrating patient demographic information and symptoms is warranted to thoroughly evaluate the potential of CD163 as a biomarker for vulnerable plaques and ⁶⁴Cu-ICT-01 PET/CT to assess the rupture index in atherosclerotic patients.

In conclusion, based on phage display screening, a CD163 targeting radiotracer of ⁶⁴Cu-ICT-01 with high binding affinity and favorable biodistribution profile was developed. In vivo PET/CT imaging in mouse atherosclerosis models revealed the sensitivity and specificity of ⁶⁴Cu-ICT-01 targeting plaques. Molecular characterization of mouse aortic arteries demonstrated the high and unique expression of CD163 on tissue resident macrophages. Ex vivo human CEA tissues characterization demonstrated the potential of CD163 as a biomarker to assess plaques rupture index and warranted further studies in future translational settings to evaluate ⁶⁴Cu-ICT-01 PET/CT to risk stratify atherosclerotic patients.

### REFERENCES

The entirety of the disclosures of the following references, and any other references disclosed herein, are incorporated herein by reference.

### REFERENCES

1. Libby P, Ridker P M, Maseri A. Inflammation and atherosclerosis. Circulation. 2002; 105:1135-1143.
2. Galkina E, Ley K. Immune and inflammatory mechanisms of atherosclerosis Annu Rev Immunol. 2009; 27:165-197.
3. Wolf D, Ley K. Immunity and Inflammation in Atherosclerosis. Circ Res. 2019; 124:315-327.
4. Li Z, Tang H., Tu Y. Molecular and Nonmolecular Imaging of Macrophages in Atherosclerosis. Front Cardiovasc Med. 2021; 8:670639.
5. Nahrendorf M, Hoyer F F, Meerwaldt A E, et al. Imaging Cardiovascular and Lung Macrophages With the Positron Emission Tomography Sensor (64)Cu-Macrin in Mice, Rabbits, and Pigs. Circ Cardiovasc Imaging. 2020; 13:e010586.
6. Kazuma S M, Sultan D, Zhao Y, et al. Recent Advances of Radionuclide-Based Molecular Imaging of Atherosclerosis. Curr Pharm Des. 2015; 21:5267-5276.
7. Li X, Rosenkrans Z T, Wang J, Cai W. PET imaging of macrophages in cardiovascular diseases. Am J Transl Res. 2020; 12:1491-1514.
8. Fang F, Xiao C, Li C, Liu X, Li S. Tuning macrophages for atherosclerosis treatment. Regenerative Biomaterials. 2022; 10.
9. Lin P, Ji H H, Li Y J, Guo S D. Macrophage Plasticity and Atherosclerosis Therapy. Front MolBiosci. 2021; 8:679797.
10. Peng R, Ji H, Jin L, et al. Macrophage-Based Therapies for Atherosclerosis Management. J Immunol Res. 2020; 2020:8131754.
11. Watanabe S, Alexander M, Misharin A V, Budinger G R S. The role of macrophages in the resolution of inflammation. J Clin Invest. 2019; 129:2619-2628.
12. Chen S, Saeed AFUH, Liu Q, et al. Macrophages in immunoregulation and therapeutics. Signal Transduction and Targeted Therapy. 2023; 8:207.
13. Park I, Kassiteridi C, Monaco C. Functional diversity of macrophages in vascular biology and disease. Vascul Pharmacol. 2017; 99:13-22.
14. Willemsen L, de Winther M P. Macrophage subsets in atherosclerosis as defined by single-cell technologies. J Pathol. 2020; 250:705-714.
15. Italiani P, Boraschi D. From Monocytes to M1/M2 Macrophages: Phenotypical vs. Functional Differentiation. Front Immunol. 2014; 5:514.
16. Dick S A, Wong A, Hamidzada H, et al. Three tissue resident macrophage subsets coexist across organs with conserved origins and life cycles. Sci Immunol. 2022; 7:eabf7777.
17. Heo G S, Sultan D, Liu Y. Current and novel radiopharmaceuticals for imaging cardiovascular inflammation. Q J Nucl Med Mol Imaging. 2020; 64:4-20.
18. Williams J W, Elvington A, Ivanov S, et al. Thermoneutrality but Not UCP1 Deficiency Suppresses Monocyte Mobilization Into Blood. Circ Res. 2017; 121:662-676.
19. Liu Y, Luehmann H P, Detering L, et al. Assessment of Targeted Nanoparticle Assemblies for Atherosclerosis Imaging with Positron Emission Tomography and Potential for Clinical Translation. ACS Appl Mater Interfaces. 2019; 11:15316-15321.
20. Sultan D, Li W, Detering L, et al. Assessment of ultrasmall nanocluster for early and accurate detection of atherosclerosis using positron emission tomography/computed tomography. Nanomedicine. 2021; 36:102416.
21. Varasteh Z, Mohanta S, Li Y, et al. Targeting mannose receptor expression on macrophages in atherosclerotic plaques of apolipoprotein E-knockout mice using (68)Ga-NOTA-anti-MMR nanobody: non-invasive imaging of atherosclerotic plaques. FJNMMI Res. 2019; 9:5.
22. Kim E J, Kim S, Seo H S, et al. Novel PET Imaging of Atherosclerosis with 68Ga-Labeled NOTA-Neomannosylated Human Serum Albumin. J Nucl Med. 2016; 57:1792-1797.
23. Derlin T, Sedding D G, Dutzmann J, et al. Imaging of chemokine receptor CXCR4 expression in culprit and nonculprit coronary atherosclerotic plaque using motion-corrected [(68)Ga]pentixafor PET/CT. Eur J Nucl Med Mol Imaging. 2018; 45:1934-1944.
24. Tarkin J M, Joshi F R, Evans N R, et al. Detection of Atherosclerotic Inflammation by (68)Ga-DOTATATE PET Compared to [(18)F]FDG PET Imaging. J Am Coll Cardiol. 2017; 69:1774-1791.
25. Douhi A, Al-Enezi M S, Berrahmoune N, et al. Non-calcified active atherosclerosis plaque detection with 18F-NaF and 18F-FDG PET/CT dynamic imaging. Phys Eng Sci Med.
2023; 46:295-302.
26. Sakamoto A, Kawakami R, Mori M, et al. CD163+ macrophages restrain vascular calcification, promoting the development of high-risk plaque. JCIInsight. 2023; 8.
27. Gutierrez-Mufloz C, Mendez-Barbero N, Svendsen P, et al. CD163 deficiency increases foam cell formation and plaque progression in atherosclerotic mice. Faseb j. 2020; 34:14960-14976.
28. Guo L, Akahori H, Harari E, et al. CD163+ macrophages promote angiogenesis and vascular permeability accompanied by inflammation in atherosclerosis. J Clin Invest. 2018; 128:1106-1124.
29. Yu J H, Kwak H S, Chung G H, Hwang S B, Park M S, Park S H. Association of Intraplaque Hemorrhage and Acute Infarction in Patients With Basilar Artery Plaque. Stroke. 2015; 46:2768-2772.
30. Stoger J L, Gijbels M J, van der Velden S, et al. Distribution of macrophage polarization markers in human atherosclerosis. Atherosclerosis. 2012; 225:461-468.

What is claimed is:

1. A CD163 targeting and theranostic composition, the composition comprising a targeting peptide comprising an amino acid sequence, a chelator and a theranostic agent; wherein the amino acid sequence is any one of SEQ ID NO. 1, SEQ ID NO. 2, SEQ ID NO. 3, or SEQ ID NO. 4.

2. The composition of claim 1, wherein the chelator is NODAGA.

3. The composition of claim 1, wherein the theranostic agent is ⁶⁴Cu.

4. The composition of claim 1, wherein the targeting peptide and chelator are covalently linked and the theranostic agent is a radionuclide.

5. The composition of claim 1, wherein the amino acid sequence is SEQ ID NO. 1, the chelator is NODAGA, and the theranostic agent is ⁶⁴Cu.

6. A method of detecting CD163 in a subject in need thereof, the method comprising: administering to the subject a composition comprising the CD163 targeting and theranostic composition of claim 1 and detecting the presence, amount, and/or location of the theranostic agent, wherein detecting the theranostic agent is indicative of CD163.

7. The method of claim 6, wherein the amino acid sequence comprises SEQ ID NO. 1, SEQ ID NO. 2, SEQ ID NO. 3, or SEQ ID NO. 4.

8. The method of claim 6, wherein the chelator is NODAGA.

9. The method of claim 6, wherein the theranostic agent is ⁶⁴Cu.

10. The method of claim 6, wherein the targeting peptide and chelator are covalently linked and the theranostic agent is a radionuclide.

11. A method of detecting activated macrophages in a subject, the method comprising: administering to the subject a composition comprising the CD163 targeting and theranostic composition of claim 1 and detecting the presence, amount, and/or location of the theranostic agent, wherein detecting the theranostic agent is indicative of activated macrophages.

12. The method of claim 11, wherein the amino acid sequence comprises SEQ ID NO. 1, SEQ ID NO. 2, SEQ ID NO. 3, or SEQ ID NO. 4.

13. The method of claim 11, wherein the chelator is NODAGA.

14. The method of claim 11, wherein the theranostic agent is ⁶⁴Cu.

15. The composition of claim 11, wherein the targeting peptide and chelator are covalently linked and the theranostic agent is a radionuclide.

16. A method of detecting inflammation in a subject, the method comprising: administering to the subject a composition comprising the CD163 targeting and theranostic composition of claim 1 and detecting the presence, amount, and/or location of the theranostic agent, wherein detecting the theranostic agent is indicative of inflammation.

17. The method of claim 16, wherein the amino acid sequence comprises SEQ ID NO. 1, SEQ ID NO. 2, SEQ ID NO. 3, or SEQ ID NO. 4.

18. The method of claim 16, wherein the chelator is NODAGA.

19. The method of claim 16, wherein the theranostic agent is ⁶⁴Cu.

20. The composition of claim 16, wherein the targeting peptide and chelator are covalently linked and the theranostic agent is a radionuclide.

21. A method of detecting and/or monitoring atherosclerotic plaques in a subject, the method comprising:
administering to the subject a composition comprising the CD163 targeting and theranostic composition of claim 1 and detecting the presence, amount, and/or location of the theranostic agent, wherein detecting the theranostic agent is indicative of atherosclerotic plaques.

22. The method of claim 21, wherein the amino acid sequence comprises SEQ ID NO. 1, SEQ ID NO. 2, SEQ ID NO. 3, or SEQ ID NO. 4.

23. The method of claim 21, wherein the chelator is NODAGA.

24. The method of claim 21, wherein the theranostic agent is ⁶⁴Cu.

25. The method of claim 21, wherein the targeting peptide and chelator are covalently linked and the theranostic agent is a radionuclide.

26. A method of treating a cancer in a subject in need thereof, comprising administering to the subject an CD163 targeting peptide comprising an amino acid sequence, a chelator, and a theranostic agent, wherein
the amino acid sequence comprises the sequence set forth in any one of SEQ ID NO. 1, SEQ ID NO. 2, SEQ ID NO. 3, or SEQ ID NO. 4;
the theranostic agent is ²²⁵At; and
the cancer is a CD163 positive cancer.

27. The method of claim 26, wherein the cancer is a solid tumor.

28. The method of claim 27, wherein the cancer comprises ovarian cancer, melanoma, hepatocellular carcinoma, colorectal cancer, head and neck cancer, pancreatic cancer, breast cancer, stomach cancer, lung cancer, prostate cancer, colon cancer, brain cancer, or myloma.


SEQUENCE LISTING (4 sequences)

SEQ ID NO: 1 - protein, 12 aa, Homo sapiens
VDDHYGTIVRNG

SEQ ID NO: 2 - protein, 12 aa, Homo sapiens
HWKWLKPWAVNT

SEQ ID NO: 3 - protein, 12 aa, Homo sapiens
LIPAPGGTPRAG

SEQ ID NO: 4 - protein, 12 aa, Homo sapiens
WFWNHGWQWSSK